SEQUENCE LISTING (9 sequences)

SEQ ID NO: 1 - protein, 547 aa, Artificial Sequence
  Wild-type human IL-12
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 219 aa, Artificial Sequence
  IL-12 alpha
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, Artificial Sequence
  IL-12 beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 532 aa, Artificial Sequence
  IL-12 amino acid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 539 aa, Artificial Sequence
  IL-12 amino acid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSSGGGGSPGGGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - RNA, 1596 nt, Artificial Sequence
  mRNA coding IL-12
augugccaccagcagcuggugaucagcugguucagccugguguuccuggccagcccccugguggccaucugggagcugaagaaggacguguacgugguggaguuggauugguaccccgacgcccccggcgagaugguggugcugaccugcgacacccccgaggaggacggcaucaccuggacccuggaccagagcagcgaggugcugggcagcggcaagacccugaccauccaggugaaggaguucggcgacgccggccaguacaccugccacaagggcggcgaggugcugagccacagccugcugcugcugcacaagaaggaggacggcaucuggagcaccgacauccugaaggaccagaaggagcccaagaacaagaccuuccugagaugcgaggccaagaacuacagcggcagauucaccugcugguggcugaccaccaucagcaccgaccugaccuucagcgugaagagcagcagaggcagcagcgacccccagggcgugaccugcggcgccgccacccugagcgccgagagagugagaggcgacaacaaggaguacgaguacagcguggagugccaggaagauagcgccugccccgccgccgaggagagccugcccaucgaggugaugguggacgccgugcacaagcugaaguacgagaacuacaccagcagcuucuucaucagagauaucaucaagcccgacccccccaagaaccugcagcugaagccccugaagaacagccggcagguggaggugagcugggaguaccccgacaccuggagcaccccccacagcuacuucagccugaccuucugcgugcaggugcagggcaagagcaagagagagaagaaagauagaguguucaccgacaagaccagcgccaccgugaucugcagaaagaacgccagcaucagcgugagagcccaagauagauacuacagcagcagcuggagcgagugggccagcgugcccugcagcggcggcggcggcggcggcagcagaaaccugcccguggccacccccgaccccggcauguuccccugccugcaccacagccagaaccugcugagagccgugagcaacaugcugcagaaggcccggcagacccuggaguucuaccccugcaccagcgaggagaucgaccacgaagauaucaccaaagauaagaccagcaccguggaggccugccugccccuggagcugaccaagaacgagagcugccugaacagcagagagaccagcuucaucaccaacggcagcugccuggccagcagaaagaccagcuucaugauggcccugugccugagcagcaucuacgaggaccugaagauguaccagguggaguucaagaccaugaacgccaagcugcugauggaccccaagcggcagaucuuccuggaccagaacaugcuggccgugaucgacgagcugaugcaggcccugaacuucaacagcgagaccgugccccagaagagcagccuggaggagcccgacuucuacaagaccaagaucaagcugugcauccugcugcacgccuucagaaucagagccgugaccaucgacagagugaugagcuaccugaacgccagc

SEQ ID NO: 7 - RNA, 1623 nt, Artificial Sequence
  mRNA coding IL-12
augugucaccagcaguuggucaucucuugguuuucccugguuuuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaaaacccugaccauccaagucaaagaguuuggagaugcuggccaguacaccugucacaaaggaggcgagguucuaagccauucgcuccugcugcuucacaaaaaggaagauggaauuugguccacugauauuuuaaaggaccagaaagaacccaaaaauaagaccuuucuaagaugcgaggccaagaauuauucuggacguuucaccugcugguggcugacgacaaucaguacugauuugacauucagugucaaaagcagcagagggucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugaaaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagaaaagaaagauagagucuucacggacaagaccucagccacggucaucugccgcaaaaaugccagcauuagcgugcgggcccaggaccgcuacuauagcucaucuuggagcgaaugggcaucugugcccugcaguggcucuagcggagggggaggcucuccuggcgggggaucuagcagaaaccuccccguggccacuccagacccaggaauguucccaugccuucaccacucccaaaaccugcugagggccgucagcaacaugcuccagaaggccagacaaacucuagaauuuuacccuugcacuucugaggaaauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugauagagugaugagcuaucugaaugcuuccugauga

SEQ ID NO: 8 - DNA, 1890 bp, Artificial Sequence
  mRNA with regulator elements
ggggaaataagagagaaaagaagagtaagaagaaatataagagccaccatgtgccaccagcagctggtgatcagctggttcagcctggtgttcctggccagccccctggtggccatctgggagctgaagaaggacgtgtacgtggtggagttggattggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgagatgcgaggccaagaactacagcggcagattcacctgctggtggctgaccaccatcagcaccgacctgaccttcagcgtgaagagcagcagaggcagcagcgacccccagggcgtgacctgcggcgccgccaccctgagcgccgagagagtgagaggcgacaacaaggagtacgagtacagcgtggagtgccaggaagatagcgcctgccccgccgccgaggagagcctgcccatcgaggtgatggtggacgccgtgcacaagctgaagtacgagaactacaccagcagcttcttcatcagagatatcatcaagcccgacccccccaagaacctgcagctgaagcccctgaagaacagccggcaggtggaggtgagctgggagtaccccgacacctggagcaccccccacagctacttcagcctgaccttctgcgtgcaggtgcagggcaagagcaagagagagaagaaagatagagtgttcaccgacaagaccagcgccaccgtgatctgcagaaagaacgccagcatcagcgtgagagcccaagatagatactacagcagcagctggagcgagtgggccagcgtgccctgcagcggcggcggcggcggcggcagcagaaacctgcccgtggccacccccgaccccggcatgttcccctgcctgcaccacagccagaacctgctgagagccgtgagcaacatgctgcagaaggcccggcagaccctggagttctacccctgcaccagcgaggagatcgaccacgaagatatcaccaaagataagaccagcaccgtggaggcctgcctgcccctggagctgaccaagaacgagagctgcctgaacagcagagagaccagcttcatcaccaacggcagctgcctggccagcagaaagaccagcttcatgatggccctgtgcctgagcagcatctacgaggacctgaagatgtaccaggtggagttcaagaccatgaacgccaagctgctgatggaccccaagcggcagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcagaatcagagccgtgaccatcgacagagtgatgagctacctgaacgccagctgataataggctggagcctcggtggccatgcttcttgccccttgggcctccccccagcccctcctccccttcctgcacccgtaccccccaaacaccattgtcacactccagtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 9 - RNA, 1623 nt, Artificial Sequence
  mRNA with regulator elements
augugucaccagcagcuggugaucucaugguucucccugguauuucuggcaucuccucuugucgcaaucugggaacugaagaaagacguguaugucguugagcucgacugguauccggaugcgccuggcgagaugguggugcugaccugugacaccccagaggaggaugggaucacuuggacccuugaucaauccuccgaagugcucgggucuggcaagacucugaccauacaagugaaagaguuuggcgaugccgggcaguacacuugccauaagggcggagaaguucugucccacucacugcugcugcugcacaagaaagaggacggaauuuggaguaccgauauccugaaagaucagaaagagcccaagaacaaaaccuucuugcggugcgaagccaagaacuacucagggagauuuacuuguugguggcugacgacgaucagcaccgaucugacuuucuccgugaaaucaaguaggggaucaucugacccucaaggagucacauguggagcggcuacucugagcgcugaacgcguaagaggggacaauaaggaguacgaguauagcguugagugccaagaggauagcgcaugccccgccgccgaagaaucauugcccauugaagugaugguggaugcuguacacaagcugaaguaugagaacuacacaagcuccuucuucauccgugacaucaucaaaccagauccuccuaagaaccuccagcuuaaaccucugaagaacucuagacagguggaagugucuugggaguaucccgacaccuggucuacaccacauuccuacuucagucucacauucugcguucagguacagggcaaguccaaaagggagaagaaggaucgggucuuuacagauaaaacaagugccaccguuauaugccggaagaaugccucuauuucugugcgugcgcaggacagauacuauagcagcucuuggagugaaugggccagugucccauguucagggucauccggugguggcggcagccccggaggcgguagcuccagaaaucucccuguggcuacaccugauccaggcauguuucccuguuugcaccauagccaaaaccuccugagagcagucagcaacaugcuccagaaagcuagacaaacacuggaauucuacccaugcaccuccgaggaaauagaucacgaggauaucacuaaggacaaaacaagcacugucgaagcaugccuucccuuggaacugacaaagaacgagaguugccuuaauucaagagaaacaucuuucauuacaaacgguagcugcuuggcaagcagaaaaacaucuuuuaugauggcccuuugucugagcaguauuuaugaggaucucaaaauguaccagguggaguuuaagaccaugaaugccaagcugcugauggacccaaagagacagauuuuccucgaucagaauaugcuggcugugauugaugaacugaugcaggccuugaauuucaacagcgaaaccguuccccagaaaagcagucuugaagaaccugacuuuuauaagaccaagaucaaacuguguauucuccugcaugccuuuagaaucagagcagucacuauagauagagugauguccuaccugaaugcuuccugauga

(19) United States
(12) Patent Application Publication
(10) Pub. No.: US 2023/0295257 A1
(43) Pub. Date: Sep. 21, 2023

(54) LIPID NANOPARTICLE COMPRISING MODIFIED NUCLEOTIDES

(71) Applicant: STRAND THERAPEUTICS INC., Boston, MA (US)

(72) Inventors: Jacob BECRAFT, Boston, MA (US); Ryan SOWELL, Boston, MA (US); Jaspreet KHURANA, Cambridge, MA (US); Tasuku KITADA, Cambridge, MA (US)

(73) Assignee: STRAND THERAPEUTICS INC., Boston, MA (US)

(21) Appl. No.: 18/006,555

(22) PCT Filed: Jul. 26, 2021
(86) PCT No.: PCT/US2021/043214

§ 371 Date: Jan. 23, 2023

Related U.S. Application Data
(60) Provisional application No. 63/056,382, filed on Jul. 24, 2020.

(51) Int. Cl. C07K 14/54; A61K 9/127; A61K 48/00; A61K 9/51; A61P 35/00

(52) U.S. Cl. CPC C07K 14/5434; A61K 9/1272; A61K 48/0041; A61K 48/0066; A61K 9/51; A61P 35/00

(57) ABSTRACT

The present disclosure relates to a lipid nanoparticle comprising (i) one or more types of lipid; and (ii) a modified mRNA comprising a sequence that encodes an interleukin (IL)-12 molecule, wherein the lipid nanoparticle is capable of triggering immunogenic cell death, and methods of treatment using the same

### CROSS-REFERENCE TO RELATED APPLICATION

This application claims priority benefit of U.S. Provisional Application No. 63/056,382, filed Jul. 24, 2020, which is herein incorporated by reference in its entirety.

### REFERENCE TO SEQUENCE LISTING SUBMITTED ELECTRONICALLY VIA EFS-WEB

The content of the electronically submitted sequence listing (Name: 4597_004PC01_Seqlisting_ST25.txt; Size: 28,633 bytes; and Date of Creation: Jul. 21, 2021) is herein incorporated by reference in its entirety.

### BACKGROUND OF THE INVENTION

Due to its ability to activate both NK cells and cytotoxic T cells, IL12 protein has been studied as a promising anti-cancer therapeutic since 1994. See Nastala, C. L. et al., J Immunol 153: 1697-1706 (1994). But despite high expectations, early clinical studies did not yield satisfactory results. Lasek W. et al., Cancer Immunol Immunother 63: 419-435, 424 (2014). Repeated administration of IL12, in most patients, led to adaptive response and a progressive decline of IL12-induced interferon gamma (IFN-γ) levels in blood. Id. Moreover, while it was recognized that IL12-induced anti-cancer activity is largely mediated by the secondary secretion of IFNγ, the concomitant induction of IFN-γ along with other cytokines (e.g., TNF-α) or chemokines (IP-10 or MIG) by IL12 caused severe toxicity. Id.

In addition to the negative feedback and toxicity, the marginal efficacy of the IL12 therapy in clinical settings may be caused by the strong immunosuppressive environment in humans. Id. To minimize IFN-γ toxicity and improve IL12 efficacy, scientists tried different approaches, such as different dose and time protocols for IL12 therapy. See Sacco, S. et al., Blood 90: 4473-4479 (1997); Leonard, J. P. et al., Blood 90: 2541-2548 (1997); Coughlin, C. M. et al., Cancer Res. 57: 2460-2467 (1997); Asselin-Paturel, C. et al., Cancer 91: 113-122 (2001); and Saudemont, A. et al., Leukemia 16: 1637-1644 (2002). Nonetheless, these approaches have not significantly impacted patient survival. Kang, W. K., et al., Human Gene Therapy 12: 671-684 (2001). Thus, there is a need in the art for an improved therapeutic approach for using IL12 to treat tumors.

### BRIEF SUMMARY OF THE INVENTION

The present disclosure provides a lipid nanoparticle comprising (i) one or more types of lipid; and (ii) a modified mRNA comprising a sequence that encodes an interleukin (IL)-12 molecule; wherein the lipid nanoparticle is capable of triggering immunogenic cell death. In some aspects, the one or more types of lipid comprises a cationic lipid. In some aspects, the cationic lipid is a compound of Formula I:

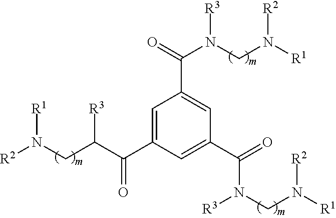

and salts thereof; wherein each R¹ is independently unsubstituted alkyl; each R² is independently unsubstituted alkyl; each R³ is independently hydrogen or substituted or unsubstituted alkyl; and each m is independently 3, 4, 5, 6, 7, or 8. In some aspects, at least one R¹ is C₁₁H₂₃. In some aspects, at least one R³ is hydrogen. In some aspects, at least one m is 3.

In some aspects, the cationic lipid is N1,N3,N5-tris(3-(didodecylamino)propyl)benzene-1,3,5-tricarboxamide (TT3), having the structure:

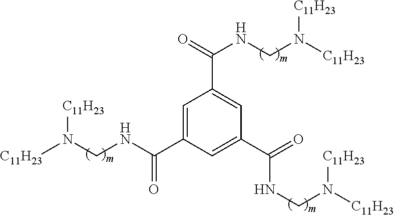

and salts thereof, wherein m is 3. In some aspects, the lipid nanoparticle comprises TT3, 1,2-Dioleoyl-sn-glycero-3-phosphoethanolamine (DOPE), cholesterol, and C14-PEG2000.

In some aspects, the modified RNA comprises a modified 5′-cap. In some aspects, the modified 5′-cap is selected from the group consisting of m₂^{7,2′-O}GppspGRNA, m⁷GpppG, m⁷Gppppm⁷G, m₂^{(7,3′-O)}GpppG, m₂^{(7,2′-O)} GppspG(D1), m₂^{(7,2′-O)} GppspG(D2), m₂^{7,3′-O}Gppp(m₁^{2′-O})ApG, (m⁷G-3′ mppp-G; which may equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, and combinations thereof.

In some aspects, the modified RNA is circular RNA.

In some aspects, the modified RNA further comprises a half-life extending moiety.

In some aspects, the half-life extending moiety comprises an Fc, an albumin or a fragment thereof, an albumin binding moiety, a PAS sequence, a HAP sequence, transferrin or a fragment thereof, an XTEN, or any combinations thereof.

In some aspects, the IL-12 molecule is selected from the group consisting of IL-12, an IL-12 subunit, or a mutant IL-12 molecule that retains the immunomodulatory function. In some aspects, the IL-12 comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 1. In some aspects, the IL-12 molecule comprises IL-12a and/or IL-120 subunits. In some aspects, the IL-12a subunit comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 2. In some aspects, the IL-120 subunit comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 3.

In some aspects, the IL-12a subunit and the IL-120 subunit are linked by a linker. In some aspects, the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids. In some aspects, the linker comprises a (GS) linker. In some aspects, the GS linker has a formula of (Gly4Ser)n or S(Gly4Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100. In some aspects, the (Gly4Ser)n linker is (Gly4Ser)3 or (Gly4Ser)4.

In some aspects, the IL12 molecule comprises an amino acid sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 4 or SEQ ID NO: 5. In some aspects, the modified mRNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 6. In some aspects, the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 7.

In some aspects, the modified RNA further comprises a regulatory element. In some aspects, the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, and combinations thereof. In some aspects, the 3′ tailing region of linked nucleosides comprises a poly-A tail, a polyA-G quartet, or a stem loop sequence.

In some aspects, the modified RNA comprises at least one modified nucleoside. In some aspects, the at least one modified nucleoside is selected from the group consisting of 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 8. In some aspects, the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 9.

In some aspects, the lipid nanoparticle has a diameter of about 30-500 nm. In some aspects, the lipid nanoparticle has a diameter of about 50-400 nm. In some aspects, the lipid nanoparticle has a diameter of about 70-300 nm. In some aspects, the lipid nanoparticle has a diameter of about 100-200 nm. In some aspects, the lipid nanoparticle has a diameter of about 100-175 nm. In some aspects, the lipid nanoparticle has a diameter of about 100-120 nm. In some aspects, the lipid nanoparticle and the modified RNA have a mass ratio of about 1:2 to about 2:1. In some aspects, the lipid nanoparticle and the modified RNA have a mass ration of 1:2, 1:1.5, 1:1.2, 1:1.1, 1:1, 1.1:1, 1.2:1, 1.5:1, or 2:1. In some aspects, the lipid and the modified RNA have a mass ratio of about 1:1.

The prevent disclosure also provides a pharmaceutical composition, comprising the lipid nanoparticle disclosed herein and a pharmaceutically acceptable carrier. In some aspects, the pharmaceutical composition is formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

The present disclosure also includes a method for treating cancer in a subject in need thereof, comprising administering to the subject an effective amount of the lipid nanoparticle or the pharmaceutical composition disclosed herein. In some aspects, the subject is a human patient having or suspected of having a cancer. In some aspects, the human patient has a cancer selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and head and neck cancer. In some aspects, the lipid nanoparticle or the pharmaceutical composition is administered to the subject in a single dose. In some aspects, the pharmaceutical composition is administered to the subject by intratumoral injection, intramuscular injection, subcutaneous injection, or intravenous injection.

### BRIEF DESCRIPTION OF THE DRAWINGS/FIGURES

FIG. 1A shows IL-12 expression level between self-replicating mRNA and modified RNA. Y axis shows the expression level in B16.F10 cell line and the X axis shows hours post transfection. FIG. 1B shows IL-12 expression level between self-replicating mRNA and modified RNA. Y axis shows the expression level in 4T1 cell line.

FIG. 2A shows the efficacy (tumor size) of modified RNA and self replicating RNA in a highly difficult to treat mouse model. The top line is for control, and the bottom two lines are for modRNA-IL-12 and repRNA-IL-12. FIG. 2B shows the efficacy (tumor size) between modified RNA-IL-12 and repRNA-IL-12.

FIG. 3 shows IL-12 payload expression in the modified RNA and replicating RNA.

FIG. 4 shows probability of survival of mice administered modRNA-IL12 or repRNA-IL12 after B16-F10 cells were introduced subcutaneously. The mRNAs were dosed as a single dose at about 350 mm³ tumor size.

### DETAILED DESCRIPTION OF THE INVENTION

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

“Polynucleotide” or “nucleic acid” as used herein means a sequence of nucleotides connected by phosphodiester linkages. Polynucleotides are presented herein in the direction from the 5′ to the 3′ direction. A polynucleotide of the present disclosure can be a deoxyribonucleic acid (DNA) molecule or ribonucleic acid (RNA) molecule. Nucleotide bases are indicated herein by a single letter code: adenine (A), guanine (G), thymine (T), cytosine (C), inosine (I) and uracil (U).

As used herein, the term “polypeptide” encompasses both peptides and proteins, unless indicated otherwise.

The term “coding sequence” or sequence “encoding” is used herein to mean a DNA or RNA region (the transcribed region) which “encodes” a particular protein, e.g., such as an IL-12. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

A Kozak consensus sequence, Kozak consensus or Kozak sequence, is known as a sequence which occurs on eukaryotic mRNA and has the consensus (gcc)gccRccAUGG, where R is a purine (adenine or guanine) three bases upstream of the start codon (AUG), which is followed by another “G.” In some aspects, the polynucleotide comprises a nucleic acid sequence having at least 95%, at least 99% sequence identity, or more to the Kozak consensus sequence. In some aspects, the polynucleotide comprises a Kozak consensus sequence.

The term “RNA” is used herein to mean a molecule which comprises at least one ribonucleotide residue. “Ribonucleotide” relates to a nucleotide with a hydroxyl group at the 2′-position of a β-D-ribofuranosyl group. The term comprises double-stranded RNA, single-stranded RNA, isolated RNA such as partially or completely purified RNA, essentially pure RNA, synthetic RNA, recombinantly generated RNA such as modified RNA which differs from naturally occurring RNA by addition, deletion, substitution and/or alteration of one or more nucleotides. The term “mRNA” means “messenger-RNA” and relates to a “transcript” which is generated by using a DNA template and encodes a peptide or protein. Typically, an mRNA comprises a 5′-UTR, a protein coding region and a 3′-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available. In some aspects of the disclosure, the RNA, preferably the mRNA, is modified with a 5′-cap structure.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case may be, as determined by the match between strings of such sequences.

In certain aspects, methods to determine identity are designed to give the largest match between the sequences tested. Methods to determine identity and similarity are codified in publicly available computer programs.

“Substantial homology” or “substantial similarity,” means, when referring to a nucleic acid or fragment thereof, indicates that, when optimally aligned with appropriate nucleotide insertions or deletions with another nucleic acid (or its complementary strand), there is nucleotide sequence identity in at least about 95 to 99% of the sequence.

The term “alkyl” as used herein is a branched or unbranched saturated hydrocarbon group of 1 to 24 carbon atoms, such as methyl, ethyl, n-propyl, isopropyl, n-butyl, isobutyl, pentyl, hexyl, heptyl, octyl, nonyl, decyl, dodecyl, tetracedcyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkoxy, alkenyl, alkynyl, aryl, heteroaryl, aldehyde, amino, carboxylic acid, ester, ether, halide, hydroxyl, ketone, nitro, silyl, sulfoxo, sulfonyl, sulfoxide, or thiol.

As used herein, the terms “effective amount,” “therapeutically effective amount,” and a “sufficient amount” of, e.g., a gene therapy composition comprising a polynucleotide disclosed herein, refer to a quantity sufficient to, when administered to the subject, including a human, effect beneficial or desired results, including clinical results, and, as such, an “effective amount” or synonym thereto depends on the context in which it is being applied.

The amount of a given therapeutic agent or composition will correspond to such an amount will vary depending upon various factors, such as the given agent, the pharmaceutical formulation, the route of administration, the type of disease or disorder, the identity of the subject (e.g., age, sex, and/or weight) or host being treated, and the like.

The term “half-life” relates to the period of time which is needed to eliminate half of the activity, amount, or number of molecules. In the context of the present invention, the half-life of an RNA is indicative for the stability of said RNA.

Lipid Nanoparticle

The present disclosure relates to the delivery of biologically active molecules to cells using lipid nanoparticles. In some aspects, the disclosure relates to an IL-12-expressing modified RNA or circular RNA encapsulated by lipid nanoparticles, the composition thereof, and use of the composition thereof to treat a subject having cancer or suspected of having cancer.

A lipid nanoparticle (LNP), as used herein, refers to a vesicle, such as a spherical vesicle, having a contiguous lipid bilayer. Lipid nanoparticles can be used in methods by which pharmaceutical therapies are delivered to targeted locations. Non-limiting examples of LNPs include liposomes, bolaamphihiles, solid lipid nanoparticles (SLN), nanostructured lipid carriers (NLC), and monolayer membrane structures (e.g., archaeosomes and micelles).

In some aspects, the lipid nanoparticle comprises one or more types of lipids. A lipid, as used herein, refers to a group of organic compounds that include, but are not limited to, esters of fatty acids and in some aspects are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. Non-limiting examples of lipids include triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). In some aspects, the one or more types of lipids in the LNP comprises a cationic lipid.

A cationic lipid, as used herein, refers to any of a number of lipid species that carry a net positive charge at a selected pH, such as a physiological pH. Such lipids include, but are not limited to N1,N3,N5-tris(3-(didodecylamino)propyl)benzene-1,3,5-tricarboxamide (TT3), N-(2,3-dioleoyloxy)propyl)-N,N,N-trimethylammonium chloride (DOTAP); lipofectamine; 1,2-DiLinoleyloxy-N,N-dimethylaminopropane (DLinDMA), 1,2-Dilinolenyloxy-N,N-dimethylaminopropane (DLenDMA); dioctadecyldimethylammonium (DODMA), Distearyldimethylammonium (DSDMA), N,N-dioleyl-N,N,-dimethylammonium chloride (DODAC); N-(2,3-dioleyloxy)propyl)-N,N,N-trimethylammonium chloride (DOTMA); N-N-distearyl-N,N-dimethylammonium bromide (DDAB); 3-(N—(N′,N′-dimethylaminoethane)-carbamoyl)cholesterol (DC-Chol) and N-(1,2-dimyristyloxprop-3-yl)-N,N-dimethyl-N-hydroxyethyl ammonium bromide (DMRIE).

In some aspects, the cationic lipid is represented by Formula I:

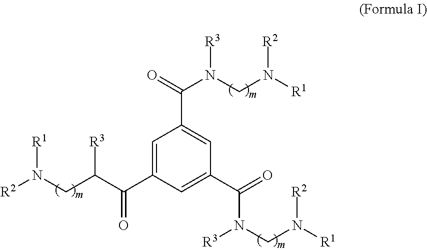

and salts thereof; wherein each R¹ is independently unsubstituted alkyl; each R² is independently unsubstituted alkyl; each R³ is independently hydrogen or substituted or unsubstituted alkyl; and each m is independently 3, 4, 5, 6, 7, or 8. In some aspects, each R¹ is independently unsubstituted alkyl; each R² is independently unsubstituted alkyl; R³ is hydrogen; and each m is 3. In some embodiments, at least one R¹ is unsubstituted C₁₋₂₄ alkyl. In some embodiments, at least one R¹ is unsubstituted C₁₋₁₈ alkyl. In some embodiments, at least one R¹ is unsubstituted C₁₋₁₂ alkyl. In some embodiments, at least one R¹ is unsubstituted C₆₋₁₈ alkyl. In some embodiments, at least one R¹ is unsubstituted C₆₋₁₂ alkyl. In some embodiments, at least one R¹ is unsubstituted C₈₋₁₂ alkyl. In some embodiments, at least one R¹ is unsubstituted C₁₀₋₁₂ alkyl. In some embodiments, at least one R¹ is unsubstituted C₁₁ alkyl.

In some aspects, at least one R² is unsubstituted C₁₋₂₄ alkyl. In some aspects, at least one R² is unsubstituted C₁₋₁₈ alkyl. In some aspects, at least one R² is unsubstituted C₁₋₁₂ alkyl. In some aspects, at least one R² is unsubstituted C₆₋₁₈ alkyl. In some aspects, at least one R² is unsubstituted C₆₋₁₂ alkyl. In some aspects, at least one R² is unsubstituted C₈₋₁₂ alkyl. In some aspects, at least one R² is unsubstituted C₁₀₋₁₂ alkyl. In some aspects, at least one R² is unsubstituted C₁₁ alkyl.

In some aspects, at least two R¹ are unsubstituted C₁₋₂₄ alkyl. In some aspects, at least two R¹ are unsubstituted C₁₋₁₈ alkyl. In some aspects, at least two R¹ are unsubstituted C₁₋₁₂ alkyl. In some aspects, at least two R¹ are unsubstituted C₆₋₁₈ alkyl. In some aspects, at least two R¹ are unsubstituted C₆₋₁₂ alkyl. In some aspects, at least two R¹ are unsubstituted C₈₋₁₂ alkyl. In some aspects, at least two R¹ are unsubstituted C₁₀₋₁₂ alkyl. In some aspects, at least two R¹ are unsubstituted C₁₁ alkyl.

In some aspects, at least two R² are unsubstituted C₁₋₂₄ alkyl. In some aspects, at least two R² are unsubstituted C₁₋₁₈ alkyl. In some aspects, at least two R² are unsubstituted C₁₋₁₂ alkyl. In some aspects, at least two R² are unsubstituted C₆₋₁₈ alkyl. In some aspects, at least two R² are unsubstituted C₆₋₁₂ alkyl. In some aspects, at least two R² are unsubstituted C₈₋₁₂ alkyl. In some aspects, at least two R² are unsubstituted C₁₀₋₁₂ alkyl. In some aspects, at least two R² are unsubstituted C₁₁ alkyl.

In some aspects, all instances of R¹ are unsubstituted C₁₋₂₄ alkyl. In some aspects, all instances of R¹ are unsubstituted C₁₋₁₈ alkyl. In some aspects, all instances of R¹ are unsubstituted C₁₋₁₂ alkyl. In some aspects, all instances of R¹ are unsubstituted C₆₋₁₈ alkyl. In some aspects, all instances of R¹ are unsubstituted C₆₋₁₂ alkyl. In some aspects, all instances of R¹ are unsubstituted C₈₋₁₂ alkyl. In some aspects, all instances of R¹ are unsubstituted C₁₀₋₁₂ alkyl. In some aspects, all instances of R¹ are unsubstituted C₁₁ alkyl.

In some aspects, all instances of R² are unsubstituted C₁₋₂₄ alkyl. In some aspects, all instances of R² are unsubstituted C₁₋₁₈ alkyl. In some aspects, all instances of R² are unsubstituted C₁₋₁₂ alkyl. In some aspects, all instances of R² are unsubstituted C₆₋₁₈ alkyl. In some aspects, all instances of R² are unsubstituted C₆₋₁₂ alkyl. In some aspects, all instances of R² are unsubstituted C₈₋₁₂ alkyl. In some aspects, all instances of R² are unsubstituted C₁₀₋₁₂ alkyl. In some aspects, all instances of R² are unsubstituted C₁₁ alkyl.

In some aspects, at least one R³ is hydrogen. In some aspects, at least one R³ is substituted or unsubstituted alkyl. In some aspects, at least one R³ is substituted or unsubstituted C₁₋₁₈ alkyl. In some aspects, at least one R³ is substituted or unsubstituted C₁₋₁₂ alkyl. In some aspects, at least one R³ is substituted or unsubstituted C₁₋₆ alkyl. In some aspects, at least one R³ is substituted or unsubstituted C₁₋₄ alkyl. In some aspects, at least one R³ is substituted or unsubstituted C₂₋₄ alkyl. In some aspects, at least one R³ is substituted or unsubstituted methyl.

In some aspects, at least one R³ is substituted alkyl, wherein the substituted alkyl is substituted with a halogen. In some aspects, at least one R³ is substituted alkyl, wherein the substituted alkyl is substituted with fluorine. In some aspects, at least one R³ is substituted alkyl, wherein the substituted alkyl is substituted with halogenated alkyl.

In some aspects, at least two R³ are hydrogen. In some aspects, at least two R³ are substituted or unsubstituted alkyl. In some aspects, at least two R³ are substituted or unsubstituted C₁₋₁₈ alkyl. In some aspects, at least two R³ are substituted or unsubstituted C₁₋₁₂ alkyl. In some aspects, at least two R³ are substituted or unsubstituted C₁₋₆ alkyl. In some aspects, at least two R³ are substituted or unsubstituted C₁₋₄ alkyl. In some aspects, at least two R³ are substituted or unsubstituted C₂₋₄ alkyl. In some aspects, at least two R³ are substituted or unsubstituted methyl.

In some aspects, at least two R³ are substituted alkyl, wherein the substituted alkyl is substituted with a halogen. In some aspects, at least two R³ are substituted alkyl, wherein the substituted alkyl is substituted with fluorine. In some aspects, at least two R³ are substituted alkyl, wherein the substituted alkyl is substituted with halogenated alkyl.

In some aspects, all instances of R³ are hydrogen. In some aspects, all instances of R³ are substituted or unsubstituted alkyl. In some aspects, all instances of R³ are substituted or unsubstituted C₁₋₁₈ alkyl. In some aspects, all instances of R³ are substituted or unsubstituted C₁₋₁₂ alkyl. In some aspects, all instances of R³ are substituted or unsubstituted C₁₋₆ alkyl. In some aspects, all instances of R³ are substituted or unsubstituted C₁₋₄ alkyl. In some aspects, all instances of R³ are substituted or unsubstituted C₂₋₄ alkyl. In some aspects, all instances of R³ are substituted or unsubstituted methyl.

In some aspects, all instances of R³ are substituted alkyl, wherein the substituted alkyl is substituted with a halogen. In some aspects, all instances of R³ are substituted alkyl, wherein the substituted alkyl is substituted with fluorine. In some aspects, all instances of R³ are substituted alkyl, wherein the substituted alkyl is substituted with halogenated alkyl.

In some aspects, at least one m is 3. In some aspects, at least one m is 4. In some aspects, at least one m is 5. In some aspects, at least one m is 6. In some aspects, at least one m is 7. In some aspects, at least one m is 8. In some aspects, at least two m are 3. In some aspects, at least two m are 4. In some aspects, at least two m are 5. In some aspects, at least two m are 6. In some aspects, at least two m are 7. In some aspects, at least two m are 8.

In some aspects, all instances of m are 3. In some aspects, all instances of m are 4. In some aspects, all instances of m are 5. In some aspects, all instances of m are 6. In some aspects, all instances of m are 7. In some aspects, all instances of m are 8.

In some aspects of the disclosure, the cationic lipid is TT3, which is represented by:

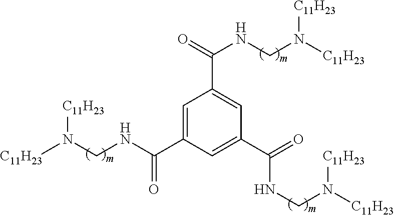

wherein all instances of m=3. The composition, synthesis, and use of Formula I and TT3 are described in WO2016187531A1, which is incorporated herein by reference.

TT3, as used herein, is capable of forming lipid nanoparticles for delivery of various biologic active agents into the cells. In addition, the present disclosure also demonstrates that an unloaded TT3-LNP can induce immunogenic cell death (ICD) in cancer cells in vivo and in vitro. Immunogenic cell death, as described herein, refers to a form of cell death that can induce an effective immune response through activation of dendritic cells (DCs) and consequent activation of specific T cell response. In some aspects of the disclosure, the cells that undergo immunogenic cell death are tumor cells. Immunogenic tumor cell death can trigger an effective anti-tumor immune response. In some aspects of the disclosure, the lipid nanoparticle comprises TT3-LNP encapsulating a modified RNA (modRNA) encoding only a reporter gene (TT3-LNP-modRNA). The modified RNA can work synergistically with the TT3-LNP to induce higher level of ICD in tumor cells compared to TT3-LNP alone. In some aspects of the disclosure, the lipid nanoparticle comprises a TT3-LNP encapsulating a modRNA encoding an IL-12 molecule. IL-12, which is an immunoregulatory cytokine, elicits a potent immune response against the local tumor. The combination of TT3-LNP, modRNA, and IL-12 expression, not only is effective in synergistic inhibition of tumor cells on site, but also elicits a systemic anti-tumor immune response to kill distal tumor cells and prevent the recurrence of tumors.

In some aspects of the disclosure, the cationic lipid is DOTAP. DOTAP, as used herein, is also capable of forming lipid nanoparticles. DOTAP can be used for the highly efficient transfection of DNA including yeast artificial chromosomes (YACs) into eukaryotic cells for transient or stable gene expression, and is also suitable for the efficient transfer of other negatively charged molecules, such as RNA, oligonucleotides, nucleotides, ribonucleoprotein (RNP) complexes, and proteins into research samples of mammalian cells.

In some aspects of the disclosure, the cationic lipid is lipofectamine. Lipfectamine, as used herein, is a common transfection reagent, produced and sold by Invitrogen, used in molecular and cellular biology. It is used to increase the transfection efficiency of RNA (including mRNA and siRNA) or plasmid DNA into in vitro cell cultures by lipofection. Lipofectamine contains lipid subunits that can form liposomes or lipid nanoparticles in an aqueous environment, which entrap the transfection payload, e.g. modRNA. The RNA-containing liposomes (positively charged on their surface) can fuse with the negatively charged plasma membrane of living cells, due to the neutral co-lipid mediating fusion of the liposome with the cell membrane, allowing nucleic acid cargo molecules to cross into the cytoplasm for replication or expression.

In some aspects of the disclosure, LNPs are composed primarily of cationic lipids along with other lipid ingredients. These typically include other lipid molecules belonging but not limited to phophatidylcholines (PC) (e.g., 1,s-Distearoyl-sn-glycero-3-phophocholine (DSPC), and 1,2-Dioleoyl-sn-glycero-3-phophoethanolamines (DOPE), sterols (e.g., cholesterol), and Polyethylene glycol (PEG)-lipid conjugates (e.g., 1,2-distearoyl-sn-glycero-3-phosphoethanolamine-N-[folate(polyethylene glycol)-2000 (DSPE-PEG2000) and 1,2-dimyristoyl-sn-glycero-3-phosphoethanolamine-N-[methoxy(polyethylene glycol)-2000 (C14-PEG2000)). Table 1 shows the formulation of exemplary LNPs, TT3-LNP and DOTAP-LNP.

TABLE 1

| DOTAP-LNP | DOTAP | DSPC | Cholesterol | DSPE-PEG2000 |
|---|---|---|---|---|
| Molar ratio | 40 | 10 | 48 | 2 |
| TT3-LNP | TT3 | DOPE | Cholesterol | C14-PEG2000 |
| Molar Ratio | 20 | 30 | 40 | 0.75 |

Particle size of lipid nanoparticles can affect drug release rate, bio-distribution, mucoadhesion, cellular uptake of water and buffer exchange to the interior of the nanoparticles, and protein diffusion. In some aspects of the disclosure, the diameter of the LNPs ranges from 30 to 500 nm. In some aspects of the disclosure, the diameter of the LNPs ranges from about 30 to about 500 nm, about 50 to about 400 nm, about 70 to about 300 nm, about 100 to about 200 nm, about 100 to about 175 nm, or about 100 to about 120 nm. In some aspects of the disclosure, the diameter of the LNPs ranges from 100-120 nm. In some aspects of the disclosure, the diameter of the LNPs can be 30 nm, 40 nm, 50 nm, 60 nm, 70 nm, 80 nm, 90 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, or 120 nm.

Zeta potential is a measure of the effective electric charge on the lipid nanoparticle surface. The magnitude of the zeta potential provides information about particle stability. In some aspects of the disclosure, the zeta potential of the LNPs ranges from 3-6 my. In some aspects of the disclosure, the zeta potential of the LNPs can be 3 my, 3.1 my, 3.2 my, 3.3 my, 3.4 my, 3.5 my, 3.6 my, 3.7 my, 3.8 my, 3.9 my, 4 my, 4.1 my, 4.2 my, 4.3 my, 4.4 my, 4.5 my, 4.6 my, 4.7 my, 4.8 my, 4.9 my, 5 my, 5.1 my, 5.2 my, 5.3 my, 5.4 my, 5.5 my, 5.6 my, 5.7 my, 5.8 my, 5.9 my, or 6 my.

In some aspects, the disclosure is related to encapsulated modRNA with lipid nanoparticles. In some aspects of the disclosure, the mass ratio between the LNPs and the modRNA ranges from 1:2 to 2:1. In some aspects, the mass ratio between the LNPs and the modRNA can be 1:2, 1:1.9, 1:1.8, 1:1.7, 1:1.6, 1:1.5, 1:1.4, 1:1.3, 1:1.2, 1:1.1, 1:1, 1.1:1, 1.2:1, 1.3:1, 1.4:1, 1.5:1, 1.6:1, 1.7:1, 1.8:1, 1.9:1, or 2:1. In some aspects of the disclosure, the mass ratio between the LNPs and the modRNA can be 1:1.

Modified RNA

In some aspects, the modified RNA is a messenger RNA. As used herein, the term “messenger RNA” (mRNA) refers to any polynucleotide which encodes a polypeptide of interest and which is capable of being translated to produce the encoded polypeptide of interest in vitro, in vivo, in situ or ex vivo. In some aspects of the disclosure, the modified RNA is synthetic.

In some aspects, the modified RNA comprises a translatable region and one, two, or more than two modifications. In some aspects, the modified nucleic acid exhibits reduced degradation in a cell into which the nucleic acid is introduced, relative to a corresponding unmodified nucleic acid.

In some aspects, the modification can be located on the sugar moiety of the nucleotide. In some aspects, the modification can be located on the phosphate backbone of the nucleotide.

In some aspects, it is desirable to intracellularly degrade a modified nucleic acid introduced into the cell, for example if precise timing of protein production is desired. Thus, in some aspects, the modified RNA comprises a degradation domain, which is capable of being acted on in a directed manner within a cell.

In some aspects, the modified RNA comprises at least one of a modified 5′-cap, a half-life extending moiety, or a regulatory element.

In some aspects, the modified 5′-cap increases the stability of the RNA, increases translation efficiency of the RNA, prolongs translation of the RNA, increases total protein expression of the RNA when compared to the same RNA without the 5′-cap structure.

In some aspects, the modified RNA is cyclized (e.g., circular mRNA), or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NETS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′—NHS-ester moiety forming a new 5′-/3′-amide bond.

In the second route, T4 RNA ligase can be used to enzymatically link a 5′-phosphorylated nucleic acid molecule to the 3′-hydroxyl group of a nucleic acid forming a new phosphorodiester linkage. In an example reaction, 1 μg of a nucleic acid molecule is incubated at 37° C. for 1 hour with 1-10 units of T4 RNA ligase (New England Biolabs, Ipswich, Mass.) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme can be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some aspects, multiple distinct nucleic acids, modified RNA or primary constructs may be linked together through the 3′-end using nucleotides which are modified at the 3′-terminus. Chemical conjugation can be used to control the stoichiometry of delivery into cells. For example, the glyoxylate cycle enzymes, isocitrate lyase and malate synthase, can be supplied into HepG2 cells at a 1:1 ratio to alter cellular fatty acid metabolism. This ratio may be controlled by chemically linking nucleic acids or modified RNA using a 3′-azido terminated nucleotide on one nucleic acids or modified RNA species and a C5-ethynyl or alkynyl-containing nucleotide on the opposite nucleic acids or modified RNA species. The modified nucleotide is added post-transcriptionally using terminal transferase (New England Biolabs, Ipswich, Mass.) according to the manufacturer's protocol. After the addition of the 3′-modified nucleotide, the two nucleic acids or modified RNA species may be combined in an aqueous solution, in the presence or absence of copper, to form a new covalent linkage via a click chemistry mechanism as described in the literature.

In some aspects, more than two polynucleotides may be linked together using a functionalized linker molecule. For example, a functionalized saccharide molecule may be chemically modified to contain multiple chemical reactive groups (SH—, NH2-, N3, etc. . . . ) to react with the cognate moiety on a 3′-functionalized mRNA molecule (i.e., a 3′-maleimide ester, 3′—NHS-ester, alkynyl). The number of reactive groups on the modified saccharide can be controlled in a stoichiometric fashion to directly control the stoichiometric ratio of conjugated nucleic acid or mRNA.

In some aspects, to further enhance protein production, nucleic acids, modified RNA, polynucleotides or primary constructs of the present disclosure can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

Conjugation may result in increased stability and/or half-life and may be particularly useful in targeting the nucleic acids, modified RNA, polynucleotides or primary constructs to specific sites in the cell, tissue or organism.

In some aspects, the primary construct is designed to encode one or more polypeptides of interest or fragments thereof. A polypeptide of interest may include, but is not limited to, whole polypeptides, a plurality of polypeptides or fragments of polypeptides, which independently may be encoded by one or more nucleic acids, a plurality of nucleic acids, fragments of nucleic acids or variants of any of the aforementioned. As used herein, the term “polypeptides of interest” refers to any polypeptide which is selected to be encoded in the primary construct of the present invention. As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

As such, polynucleotides encoding polypeptides of interest containing substitutions, insertions and/or additions, deletions and covalent modifications with respect to reference sequences are included within the scope of this invention. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein can optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

As recognized by those skilled in the art, protein fragments, functional protein domains, and homologous proteins are also considered to be within the scope of polypeptides of interest of this invention. For example, provided herein is any protein fragment (meaning an polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a polypeptide to be utilized in accordance with the invention includes 2, 3, 4, 5, 6, 7, 8, 9, 10, or more mutations as shown in any of the sequences provided or referenced herein.

In some aspects, the modified RNA comprises a modified 5′-cap, a half-life extending moiety, a regulatory element, or combinations thereof.

In some aspects, the the modified 5′-cap is selected from the group consisting of m₂^{7,2′-O}GppₛpGRNA, m⁷GpppG, m⁷Gppppm⁷G, m₂^{(7′3′-O)}GpppG, m₂^{(7-2′-O)}GppspG(D1), m₂^{(7,2′-O)}GppspG(D2), m₂^{7,3′-O}Gppp(m₁^{2′-O}) ApG, (m⁷G-3′ mppp-G; which may equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, and combinations thereof.

The disclosure also includes a polynucleotide that comprises both a 5′ Cap and a modified RNA of the disclosure (e.g., a polynucleotide comprising a nucleotide sequence encoding an IL12B polypeptide, an IL12A polypeptide, and/or IL12B and IL12A fusion polypeptides).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

According to the present disclosure, 5′ terminal caps can include endogenous caps or cap analogs. According to the present disclosure, a 5′ terminal cap can comprise a guanine analog. Useful guanine analogs include, but are not limited to, inosine, Nl-methyl-guanosine, 2′fluoro-guanosine, 7-deaza-guanosine, 8-oxo-guanosine, 2-amino-guanosine, LNA-guanosine, and 2-azido-guanosine.

In some aspects, the 5′ terminal cap structure is a CapO, Capl, ARC A, inosine, Nl-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

Non-limiting additional Caps include the 5′ Caps disclosed in WO/2017/201350, published Nov. 23, 2017, which is incorporated herein by reference.

In some aspects, the half-life extending moiety comprises an Fc, an albumin or a fragment thereof, an albumin binding moiety, a PAS sequence, a HAP sequence, transferrin or a fragment thereof, an XTEN, or any combinations thereof.

In some aspects, the half-life extending moiety comprises an Fc. In some aspects, the half-life extending moiety comprises an albumin or a fragment thereof.

In some aspects, the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, and combinations thereof.

In some aspects, the regulatory element further comprises a polyA region. In some aspects, the modified RNA of the present disclosure (e.g., a polynucleotide comprising a nucleotide sequence encoding an IL12B polypeptide, an IL12A polypeptide, and/or IL12B and IL12A fusion polypeptides) further comprise a poly-A tail. In some aspects, terminal groups on the poly-A tail can be incorporated for stabilization. In other embodiments, a poly-A tail comprises des-3′ hydroxyl tails. During RNA processing, a long chain of adenine nucleotides (poly-A tail) can be added to a polynucleotide such as an mRNA molecule in order to increase stability.

Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety). See also WO/2017/201350, published Nov. 23, 2017, which is incorporated herein by reference, for additional poly-A tails.

In some aspects, the modified RNA comprises any modification or combination of modifications described herein.

Terminal Architecture Modifications: Untranslated Regions (UTRs)

Untranslated regions (UTRs) of a gene are transcribed but not translated. The 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the modified RNA of the present disclosure to enhance the stability of the molecule. The specific features can also be incorporated to ensure controlled down-regulation of the transcript in case they are misdirected to undesired organs sites.

5′ UTR and Translation Initiation

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

5′UTR secondary structures involved in elongation factor binding can interact with other RNA binding molecules in the 5′UTR or 3′UTR to regulate gene expression. For example, the elongation factor EIF4A2 binding to a secondarily structured element in the 5′UTR is necessary for microRNA mediated repression (Meijer H A et al., Science, 2013, 340, 82-85, herein incorporated by reference in its entirety). The different secondary structures in the 5′UTR can be incorporated into the flanking region to either stabilize or selectively destabilize mRNAs in specific tissues or cells.

By engineering the features typically found in abundantly expressed genes of specific target organs, one can enhance the stability and protein production of the nucleic acids or mRNA of the invention. For example, introduction of 5′ UTR of liver-expressed mRNA, such as albumin, serum amyloid A, Apolipoprotein AB/E, transferrin, alpha fetoprotein, erythropoietin, or Factor VIII, could be used to enhance expression of a nucleic acid molecule, such as a mmRNA, in hepatic cell lines or liver. Likewise, use of 5′ UTR from other tissue-specific mRNA to improve expression in that tissue is possible—for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences may be incorporated into the 5′ (or 3′ UTR) UTRs. For example, introns or portions of introns sequences may be incorporated into the flanking regions of the nucleic acids or mRNA of the invention. Incorporation of intronic sequences may increase protein production as well as mRNA levels.

In some aspects of the disclosure, at least one fragment of IRES sequences from a GTX gene may be included in the 5′UTR. As a non-limiting example, the fragment may be an 18 nucleotide sequence from the IRES of the GTX gene. As another non-limiting example, an 18 nucleotide sequence fragment from the IRES sequence of a GTX gene may be tandemly repeated in the 5′UTR of a polynucleotide described herein. The 18 nucleotide sequence may be repeated in the 5′UTR at least one, at least twice, at least three times, at least four times, at least five times, at least six times, at least seven times, at least eight times, at least nine times or more than ten times.

Nucleotides can be mutated, replaced and/or removed from the 5′ (or 3′) UTRs. For example, one or more nucleotides upstream of the start codon can be replaced with another nucleotide. The nucleotide or nucleotides to be replaced can be 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more than 60 nucleotides upstream of the start codon. As another example, one or more nucleotides upstream of the start codon can be removed from the UTR.

3′UTR and Translation Enhancer Elements (TEEs)

In some aspects, the 5′UTR of the modified RNA comprises at least one translational enhancer polynucleotide, translation enhancer element, translational enhancer elements (collectively referred to as “TEE”s). In some aspects, the TEE is located between the transcription promoter and the start codon. In some aspects, the modified RNA with at least one TEE in the 5′UTR comprises a cap at the 5′UTR. In some aspects, the at least one TEE may be located in the 5′UTR of modified RNA undergoing cap-dependent or cap-independent translation.

The term “translational enhancer element” or “translation enhancer element” (herein collectively referred to as “TEE”) refers to sequences that increase the amount of polypeptide or protein produced from an mRNA.

In one aspect, TEEs are conserved elements in the UTR which can promote translational activity of a nucleic acid such as, but not limited to, cap-dependent or cap-independent translation. The conservation of these sequences has been previously shown by Panek et al (Nucleic Acids Research, 2013, 1-10; herein incorporated by reference in its entirety) across 14 species including humans.

In some aspects, the modified RNA has at least one TEE that has at least 50%, at least 55%, at least 60%, at least 65%, at least 70%, at least 75%, at least 80%, at least 85%, at least 90%, at least 95% or at least 99% identity with the disclosed in U.S. Application Number 2014/0147454, which is hereby incorporated by reference in its entirety. In some aspects, the modified RNA includes at least one TEE that has at least 50%, at least 55%, at least 60%, at least 65%, at least 70%, at least 75%, at least 80%, at least 85%, at least 90%, at least 95% or at least 99% identity with the TEEs described in US Patent Publication Nos. US20090226470, US20070048776, US20130177581 and US20110124100, International Patent Publication No. WO1999024595, WO2012009644, WO2009075886 and WO2007025008, European Patent Publication No. EP2610341A1 and EP2610340A1, U.S. Pat. Nos. 6,310,197, 6,849,405, 7,456,273, 7,183,395, each of which is herein incorporated by reference in its entirety.

In some aspects, the 5′UTR of the modified RNA may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 5′UTR of the modified RNA are the same or different TEE sequences. In some aspects, the TEE sequences are in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the spacer separating two TEE sequences includes other sequences known in the art which regulate the translation of the modified RNA such as, but not limited to, miR sequences described herein (e.g., miR binding sites and miR seeds). In some aspects, each spacer used to separate two TEE sequences includes a different miR sequence or component of a miR sequence (e.g., miR seed sequence).

In some aspects, the TEE used in the 5′UTR of the modified RNAof the present invention is an IRES sequence such as, but not limited to, those described in U.S. Pat. No. 7,468,275 and International Patent Publication No. WO2001055369, each of which is herein incorporated by reference in its entirety.

In some aspects, the TEEs described herein are located in the 5′UTR and/or the 3′UTR of the modified RNA. In some aspects, the TEEs located in the 3′UTR are the same and/or different than the TEEs located in and/or described for incorporation in the 5′UTR.

In some aspects, the 3′UTR of the modified RNA can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In some aspects, the TEE sequences in the 3′UTR of the modified RNA of the present disclosure is the same or different TEE sequences. The TEE sequences is in a pattern such as ABABAB or AABBAABBAABB or ABCABCABC or variants thereof repeated once, twice, or more than three times. In these patterns, each letter, A, B, or C represent a different TEE sequence at the nucleotide level.

In some aspects, the 3′UTR includes a spacer to separate two TEE sequences. In some aspects, the spacer is a 15 nucleotide spacer and/or other spacers known in the art. In some aspects, the 3′UTR may include a TEE sequence-spacer module repeated at least once, at least twice, at least 3 times, at least 4 times, at least 5 times, at least 6 times, at least 7 times, at least 8 times and at least 9 times or more than 9 times in the 3′UTR.

In some aspects, the spacer separating two TEE sequences includes other sequences known in the art which regulate the translation of the modified RNA, such as, but not limited to, miR sequences described herein (e.g., miR binding sites and miR seeds). In some aspects, each spacer used to separate two TEE sequences includes a different miR sequence or component of a miR sequence (e.g., miR seed sequence).

Incorporating microRNA Binding Sites

In some aspects, the modified RNA further comprises a sensor sequence. Sensor sequences include, for example, microRNA binding sites, transcription factor binding sites, structured mRNA sequences and/or motifs, artificial binding sites engineered to act as pseudo-receptors for endogenous nucleic acid binding molecules. Non-limiting examples, of polynucleotides comprising at least one sensor sequence are described U.S. Application No. 2014/0147454, which is hereby incorporated by reference in its entirety.

In some aspects, microRNA (miRNA) profiling of the target cells or tissues is conducted to determine the presence or absence of miRNA in the cells or tissues.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. In some aspects, the modified RNA, comprises one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety. As a non-limiting example, known microRNAs, their sequences and seed sequences in human genome are described in U.S. Application No. 2014/0147454, which is herein incorporated by reference in its entirety.

A microRNA sequence comprises a “seed” region, i.e., a sequence in the region of positions 2-8 of the mature microRNA, which sequence has perfect Watson-Crick complementarity to the miRNA target sequence. A microRNA seed comprises positions 2-8 or 2-7 of the mature microRNA. In some aspects, a microRNA seed comprises 7 nucleotides (e.g., nucleotides 2-8 of the mature microRNA), wherein the seed-complementary site in the corresponding miRNA target is flanked by an adenine (A) opposed to microRNA position 1. In some aspects, a microRNA seed comprises 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105. The bases of the microRNA seed have complete complementarity with the target sequence. By engineering microRNA target sequences into the 3′UTR of nucleic acids or mRNA of the invention one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is herein incorporated by reference in its entirety).

For example, if the mRNA is not intended to be delivered to the liver but ends up there, then miR-122, a microRNA abundant in liver, can inhibit the expression of the gene of interest if one or multiple target sites of miR-122 are engineered into the 3′UTR of the modified nucleic acids, enhanced modified RNA or ribonucleic acids. Introduction of one or multiple binding sites for different microRNA can be engineered to further decrease the longevity, stability, and protein translation of a modified nucleic acids, enhanced modified RNA or ribonucleic acids. As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the modified nucleic acids, enhanced modified RNA or ribonucleic acids of the present invention, microRNA binding sites can be engineered out of (i.e. removed from) sequences in which they naturally occur in order to increase protein expression in specific tissues. For example, miR-122 binding sites may be removed to improve protein expression in the liver.

In some aspects, the modified RNA includes at least one miRNA-binding site in the 3′UTR in order to direct cytotoxic or cytoprotective mRNA therapeutics to specific cells such as, but not limited to, normal and/or cancerous cells (e.g., HEP3B or SNU449).

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, microRNAs are known to be differentially expressed in immune cells (also called hematopoietic cells), such as antigen presenting cells (APCs) (e.g. dendritic cells and macrophages), macrophages, monocytes, B lymphocytes, T lymphocytes, granuocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. It was demonstrated in the art that the immune response to exogenous nucleic acid molecules was shut-off by adding miR-142 binding sites to the 3′UTR of the delivered gene construct, enabling more stable gene transfer in tissues and cells. miR-142 efficiently degrades the exogenous mRNA in antigen presenting cells and suppresses cytotoxic elimination of transduced cells (Annoni A et al., blood, 2009, 114, 5152-5161; Brown B D, et al., Nat med. 2006, 12(5), 585-591; Brown B D, et al., blood, 2007, 110(13): 4144-4152, each of which is herein incorporated by reference in its entirety).

Many microRNA expression studies are conducted in the art to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (U52013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, U52010/0323357); cutaneous T cell lymphoma (WO2013/011378); colorectal cancer cells (WO2011/0281756, WO2011/076142); cancer positive lympho nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in their entirety.)

At least one microRNA site can be engineered into the 3′ UTR of the modified RNA. In some aspects, at least two, at least three, at least four, at least five, at least six, at least seven, at least eight, at least nine, at least ten or more microRNA sites may be engineered into the 3′ UTR of the modified RNA. In some aspects, the microRNA sites incorporated into the modified RNA are the same or different microRNA sites. In some aspects, the microRNA sites incorporated into the modified RNA targets the same or different tissues in the body. As a non-limiting example, through the introduction of tissue-, cell-type-, or disease-specific microRNA binding sites in the 3′ UTR of a modified nucleic acid mRNA, the degree of expression in specific cell types (e.g. hepatocytes, myeloid cells, endothelial cells, cancer cells, etc.) can be reduced.

In some aspects, a microRNA site is engineered near the 5′ terminus of the 3′UTR, about halfway between the 5′ terminus and 3′ terminus of the 3′UTR and/or near the 3′ terminus of the 3′UTR. In some aspects, a microRNA site is engineered near the 5′ terminus of the 3′UTR and about halfway between the 5′ terminus and 3′ terminus of the 3′UTR. In some aspects, a microRNA site is engineered near the 3′ terminus of the 3′UTR and about halfway between the 5′ terminus and 3′ terminus of the 3′UTR. In some aspects, a microRNA site is engineered near the 5′ terminus of the 3′UTR and near the 3′ terminus of the 3′UTR.

In some aspects, a modified messenger RNA comprises microRNA binding region sites that either have 100% identity to known seed sequences or have less than 100% identity to seed sequences. The seed sequence can be partially mutated to decrease microRNA binding affinity and as such result in reduced downmodulation of that mRNA transcript. In essence, the degree of match or mis-match between the target mRNA and the microRNA seed can act as a rheostat to more finely tune the ability of the microRNA to modulate protein expression. In addition, mutation in the non-seed region of a microRNA binding site may also impact the ability of a microRNA to modulate protein expression.

RNA Motifs for RNA Binding Proteins (RBPs)

RNA binding proteins (RBPs) can regulate numerous aspects of co- and post-transcription gene expression such as, but not limited to, RNA splicing, localization, translation, turnover, polyadenylation, capping, modification, export and localization. RNA-binding domains (RBDs), such as, but not limited to, RNA recognition motif (RR) and hnRNP K-homology (KH) domains, typically regulate the sequence association between RBPs and their RNA targets (Ray et al. Nature 2013. 499:172-177; herein incorporated by reference in its entirety). In some aspects, the canonical RBDs bind short RNA sequences. In some aspects, the canonical RBDs recognize RNA structure.

Non limiting examples of RNA binding proteins and related nucleic acid and protein sequences are described in U.S. Application No. 2014/0147454, which is herein incorporated by reference in its entirety.

In some aspects, to increase the stability of the mRNA of interest, an mRNA encoding HuR is co-transfected or co-injected along with the mRNA of interest into the cells or into the tissue. These proteins can also be tethered to the mRNA of interest in vitro and then administered to the cells together. Poly A tail binding protein, PABP interacts with eukaryotic translation initiation factor eIF4G to stimulate translational initiation. Co-administration of mRNAs encoding these RBPs along with the mRNA drug and/or tethering these proteins to the mRNA drug in vitro and administering the protein-bound mRNA into the cells can increase the translational efficiency of the mRNA. The same concept can be extended to co-administration of mRNA along with mRNAs encoding various translation factors and facilitators as well as with the proteins themselves to influence RNA stability and/or translational efficiency.

In some aspects, the modified RNA comprises at least one RNA-binding motif such as, but not limited to a RNA-binding domain (RBD).

In some aspects, the first region of linked nucleosides and/or at least one flanking region comprises at least on RBD. In some aspects, the first region of linked nucleosides comprises a RBD related to splicing factors and at least one flanking region comprises a RBD for stability and/or translation factors.

Other Regulatory Elements in 3′UTR

In addition to microRNA binding sites, other regulatory sequences in the 3′-UTR of natural mRNA, which regulate mRNA stability and translation in different tissues and cells, can be removed or introduced into modified messenger RNA. Such cis-regulatory elements may include, but are not limited to, Cis-RNP (Ribonucleoprotein)/RBP (RNA binding protein) regulatory elements, AU-rich element (AUE), structured stem-loop, constitutive decay elements (CDEs), GC-richness and other structured mRNA motifs (Parker B J et al., Genome Research, 2011, 21, 1929-1943, which is herein incorporated by reference in its entirety). For example, CDEs are a class of regulatory motifs that mediate mRNA degradation through their interaction with Roquin proteins. In particular, CDEs are found in many mRNAs that encode regulators of development and inflammation to limit cytokine production in macrophage (Leppek K et al., 2013, Cell, 153, 869-881, which is herein incorporated by reference in its entirety).

In some aspects, the modified mRNA is auxotrophic. As used herein, the term “auxotrophic” refers to mRNA that comprises at least one feature that triggers, facilitates or induces the degradation or inactivation of the mRNA in response to spatial or temporal cues such that protein expression is substantially prevented or reduced. Such spatial or temporal cues include the location of the mRNA to be translated such as a particular tissue or organ or cellular environment. Also contemplated are cues involving temperature, pH, ionic strength, moisture content and the like.

3′ UTR and the AU Rich Elements

3′UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids or mRNA of the invention. When engineering specific nucleic acids or mRNA, one or more copies of an ARE can be introduced to make nucleic acids or mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids or mRNA of the invention and protein production can be assayed at various time points post-transfection. For example, cells can be transfected with different ARE-engineering molecules and by using an ELISA kit to the relevant protein and assaying protein produced at 6 hr, 12 hr, 24 hr, 48 hr, and 7 days post-transfection.

3′ UTR and Triple Helices

In some aspects, the modified RNA comprises a triple helix on the 3′ end of the modified nucleic acid, enhanced modified RNA or ribonucleic acid. In some aspects, the 3′ end of the modified RNA include a triple helix alone or in combination with a Poly-A tail.

In some aspects, the modified RNA comprises at least a first and a second U-rich region, a conserved stem loop region between the first and second region and an A-rich region. In some aspects, the first and second U-rich region and the A-rich region associate to form a triple helix on the 3′ end of the nucleic acid. This triple helix may stabilize the nucleic acid, enhance the translational efficiency of the nucleic acid and/or protect the 3′ end from degradation. Exemplary triple helices include, but are not limited to, the triple helix sequence of metastasis-associated lung adenocarcinoma transcript 1 (MALAT1), MEN-β and polyadenylated nuclear (PAN) RNA (See Wilusz et al., Genes & Development 2012 26:2392-2407; herein incorporated by reference in its entirety).

Stem Loop

In some aspects, the modified RNA includes a stem loop such as, but not limited to, a histone stem loop. In some aspects, the stem loop is a nucleotide sequence that is about 25 or about 26 nucleotides in length such as, but not limited to, SEQ ID NOs: 7-17 as described in International Patent Publication No. WO2013103659, herein incorporated by reference in its entirety. The histone stem loop may be located 3′ relative to the coding region (e.g., at the 3′ terminus of the coding region). As a non-limiting example, the stem loop may be located at the 3′ end of a nucleic acid described herein.

In some aspects, the modified RNA, which comprises the histone stem loop may be stabilized by the addition of at least one chain terminating nucleoside. Not wishing to be bound by theory, the addition of at least one chain terminating nucleoside may slow the degradation of a nucleic acid and thus can increase the half-life of the nucleic acid.

In some aspects, the chain terminating nucleoside is one described in International Patent Publication No. WO2013103659, herein incorporated by reference in its entirety. In some aspects, the chain terminating nucleosides are 3′-deoxyadenosine (cordycepin), 3′-deoxyuridine, 3′-deoxycytosine, 3′-deoxyguanosine, 3′-deoxythymine, 2′,31-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or a —O-methylnucleoside.

In some aspects, the modified RNA includes a histone stem loop, a polyA tail sequence and/or a 5′ cap structure. In some aspects, the histone stem loop is before and/or after the polyA tail sequence. The nucleic acids comprising the histone stem loop and a polyA tail sequence may include a chain terminating nucleoside described herein.

In some aspects, the modified RNA comprises a histone stem loop and a 5′ cap structure. The 5′ cap structure may include, but is not limited to, those described herein and/or known in the art.

5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the RNA of the present disclosure may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′ mppp-G; which may equivalently be designated 3′ 0-Me-m7G(51)ppp(5′)G). The 3′-O atom of the other, unmodified, guanine becomes linked to the 5′-terminal nucleotide of the capped nucleic acid molecule (e.g. an mRNA or mmRNA). The N7- and 3′-O-methylated guanine provides the terminal moiety of the capped nucleic acid molecule (e.g. mRNA or mmRNA).

Another exemplary cap is mCAP, which is similar to ARCA but has a 2′-β-methyl group on guanosine (i.e., N7,2′-O-dimethyl-guanosine-5′-triphosphate-5′-guanosine, m7Gm-ppp-G).

In some aspects, the cap is a dinucleotide cap analog. In some aspects, the dinucleotide cap analog is modified at different phosphate positions with a boranophosphate group or a phosphoroselenoate group such as the dinucleotide cap analogs described in U.S. Pat. No. 8,519,110, the contents of which are herein incorporated by reference in its entirety.

In some aspects, the cap is a cap analog is a N7-(4-chlorophenoxyethyl) substituted dicucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In some aspects, a cap analog of the present invention is a 4-chloro/bromophenoxyethyl analog.

While cap analogs allow for the concomitant capping of a nucleic acid molecule in an in vitro transcription reaction, up to 20% of transcripts remain uncapped. This, as well as the structural differences of a cap analog from an endogenous 5′-cap structures of nucleic acids produced by the endogenous, cellular transcription machinery, may lead to reduced translational competency and reduced cellular stability.

In some aspects, providing an RNA with a 5′-cap or 5′-cap analog is achieved by in vitro transcription of a DNA template in the presence of said 5′-cap or 5′-cap analog, wherein said 5′-cap is co-transcriptionally incorporated into the generated RNA strand,

In some aspects, RNA may be generated, for example, by in vitro transcription, and the 5′-cap may be attached to the RNA post-transcriptionally using capping enzymes, for example, capping enzymes of vaccinia virus. In some aspects, the modified RNA is capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present invention are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. This cap results in a higher translational-competency and cellular stability and a reduced activation of cellular pro-inflammatory cytokines, as compared, e.g., to other 5′ cap analog structures known in the art. Cap structures include 7mG(5′)ppp(5′)N,pN2p, 7mG(5′)ppp(5′)NlmpNp, 7mG(5′)-ppp(5′)NlmpN2 mp and m(7)Gpppm(3)(6,6,2′)Apm(2′)Apm(2′)Cpm(2)(3,2′)Up.

In some aspects, 5′ terminal caps include endogenous caps or cap analogs. In some aspects, a 5′ terminal cap comprises a guanine analog. Useful guanine analogs include inosine, N1-methyl-guanosine, 2′ fluoro-guanosine, 7-deaza-guanosine, 8-oxo-guanosine, 2-amino-guanosine, LNA-guanosine, and 2-azido-guanosine.

In some aspects, the 5′ cap comprises a 5′ to 5′ triphosphate linkage. In some aspects, the 5′ cap comprises a 5′ to 5′ triphosphate linkage including thiophosphate modification. In some aspects, the 5′ cap comprises a 2′-O or 3′-O-ribose-methylated nucleotide. In some aspects, the 5′ cap comprises a modified guanosine nucleotide or modified adenosine nucleotide. In some aspects, the 5′ cap comprises 7-methylguanylate. Exemplary cap structures include m7G(5′)ppp(5′) G, m7,2′O-mG(5′)ppSp(5′)G, m7G(5′)ppp(5′)2′O-mG, and m7,3′O-mG(5′) ppp(5′) 2′O-mA.

In some aspects, the modified RNA comprises a modified 5′ cap. A modification on the 5′ cap may increase the stability of mRNA, increase the half-life of the mRNA, and could increase the mRNA translational efficiency. In some aspects, the modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

The 5′ cap structure that may be modified includes, but is not limited to, the caps described in U.S. Application No. 2014/0147454 and WO2018/160540 which is incorporated herein by reference in its entirety.

IRES Sequences

In some aspects, the modified RNA comprises an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. Nucleic acids or mRNA containing more than one functional ribosome binding site may encode several peptides or polypeptides that are translated independently by the ribosomes (“multicistronic nucleic acid molecules”). When nucleic acids or mRNA are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

Terminal Architecture Modifications: Poly-A Tails

During RNA processing, a long chain of adenine nucleotides (poly-A tail) is normally added to a messenger RNA (mRNA) molecules to increase the stability of the molecule. Immediately after transcription, the 3′ end of the transcript is cleaved to free a 3′ hydroxyl. Then poly-A polymerase adds a chain of adenine nucleotides to the RNA. The process, called polyadenylation, adds a poly-A tail that is between 100 and 250 residues long.

In some aspects, the length of the 3′ tail is greater than 30 nucleotides in length. In some aspects, the poly-A tail is greater than 35 nucleotides in length. In some aspects, the length is at least 40 nucleotides. In some aspects, the length is at least 45 nucleotides. In some aspects, the length is at least 55 nucleotides. In some aspects, the length is at least 60 nucleotides. In some aspects, the length is at least 60 nucleotides. In some aspects, the length is at least 80 nucleotides. In some aspects, the length is at least 90 nucleotides. In some aspects, the length is at least 100 nucleotides. In some aspects, the length is at least 120 nucleotides. In some aspects, the length is at least 140 nucleotides. In some aspects, the length is at least 160 nucleotides. In some aspects, the length is at least 180 nucleotides. In some aspects, the length is at least 200 nucleotides. In some aspects, the length is at least 250 nucleotides. In some aspects, the length is at least 300 nucleotides. In some aspects, the length is at least 350 nucleotides. In some aspects, the length is at least 400 nucleotides. In some aspects, the length is at least 450 nucleotides. In some aspects, the length is at least 500 nucleotides. In some aspects, the length is at least 600 nucleotides. In some aspects, the length is at least 700 nucleotides. In some aspects, the length is at least 800 nucleotides. In some aspects, the length is at least 900 nucleotides. In some aspects, the length is at least 1000 nucleotides. In some aspects, the length is at least 1100 nucleotides. In some aspects, the length is at least 1200 nucleotides. In some aspects, the length is at least 1300 nucleotides. In some aspects, the length is at least 1400 nucleotides. In some aspects, the length is at least 1500 nucleotides. In some aspects, the length is at least 1600 nucleotides. In some aspects, the length is at least 1700 nucleotides. In some aspects, the length is at least 1800 nucleotides. In some aspects, the length is at least 1900 nucleotides. In some aspects, the length is at least 2000 nucleotides. In some aspects, the length is at least 2500 nucleotides. In some aspects, the length is at least 3000 nucleotides.

In some aspects, the modified RNA is designed to include a polyA-G Quartet. The G-quartet is a cyclic hydrogen bonded array of four guanine nucleotides that can be formed by G-rich sequences in both DNA and RNA. In this aspect, the G-quartet is incorporated at the end of the poly-A tail. The resultant nucleic acid or mRNA may be assayed for stability, protein production and other parameters including half-life at various time points. It has been discovered that the polyA-G quartet results in protein production equivalent to at least 75% of that seen using a poly-A tail of 120 nucleotides alone.

In some aspects, the modified RNA comprises a polyA tail and is stabilized by the addition of a chain terminating nucleoside. In some aspects, the modified RNA with a polyA tail further comprise a 5′ cap structure.

In some aspects, the modified RNA comprises a polyA-G Quartet. In some aspects, the modified RNA with a polyA-G Quartet further comprises a 5′ cap structure.

In some aspects, the modified RNA, which comprise a polyA tail or a polyA-G Quartet is stabilized by the addition of an oligonucleotide that terminates in a 3′-deoxynucleoside, 2′,3′-dideoxynucleoside 3′-O-methylnucleosides, 3′-0-ethylnucleosides, 3′-arabinosides, and other modified nucleosides known in the art and/or described herein.

Modified Nucleosides

In some aspects, the modified RNA comprises one or more modified nucleosides. In some aspects, the one or more modified nucleosides comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

In some aspects, one or more uridine in the modified RNA is replaced by a modified nucleoside. In some aspects, he modified nucleoside replacing uridine is pseudouridine (ψ), N1-methyl-pseudouridine (m1ψ) or 5-methyl-uridine (m5U).

In some aspects, the modified RNA comprises a modified RNA as described in U.S. Application Number 2014/0147454, International Application WO2018160540, International Application WO2015/196118, or International Application WO2015/089511, which are incorporated herein by reference in their entirety.

Cytotoxic Nucleosides

In some aspects, the modified RNA comprises one or more cytotoxic nucleosides. For example, cytotoxic nucleosides can be incorporated into polynucleotides such as bifunctional modified RNAs or mRNAs. Cytotoxic nucleoside anti-cancer agents include, but are not limited to, adenosine arabinoside, cytarabine, cytosine arabinoside, 5-fluorouracil, fludarabine, floxuridine, FTORAFUR® (a combination of tegafur and uracil), tegafur ((RS)-5-fluoro-1-(tetrahydrofuran-2-yl)pyrimidine-2,4(1H,3H)-dione), and 6-mercaptopurine.

A number of cytotoxic nucleoside analogues are in clinical use, or have been the subject of clinical trials, as anticancer agents. Examples of such analogues include, but are not limited to, cytarabine, gemcitabine, troxacitabine, decitabine, tezacitabine, 2′-deoxy-2′-methylidenecytidine (DMDC), cladribine, clofarabine, 5-azacytidine, 4′-thio-aracytidine, cyclopentenyl cytosine and 1-(2-C-cyano-2-deoxy-beta-D-arabino-pentofuranosyl)-cytosine. Another example of such a compound is fludarabine phosphate. These compounds may be administered systemically and may have side effects which are typical of cytotoxic agents such as, but not limited to, little or no specificity for tumor cells over proliferating normal cells.

A number of prodrugs of cytotoxic nucleoside analogues are also reported in the art. Examples include, but are not limited to, N4-behenoyl-1-beta-D-arabinofuranosylcytosine, N4-octadecyl-1-beta-D-arabinofuranosyl cytosine, N4-palmitoyl-1-(2-C-cyano-2-deoxy-beta-D-arabino-pentofuranosyl) cytosine, and P-4055 (cytarabine 5′-elaidic acid ester). In general, these prodrugs may be converted into the active drugs mainly in the liver and systemic circulation and display little or no selective release of active drug in the tumor tissue. For example, capecitabine, a prodrug of 5′-deoxy-5-fluorocytidine (and eventually of 5-fluorouracil), is metabolized both in the liver and in the tumor tissue. A series of capecitabine analogues containing “an easily hydrolysable radical under physiological conditions” has been claimed by Fujiu et al. (U.S. Pat. No. 4,966,891) and is herein incorporated by reference. The series described by Fujiu includes N4 alkyl and aralkyl carbamates of 5′-deoxy-5-fluorocytidine and the implication that these compounds will be activated by hydrolysis under normal physiological conditions to provide 5′-deoxy-5-fluorocytidine.

A series of cytarabine N4-carbamates has been by reported by Fadl et al (Pharmazie. 1995, 50, 382-7, herein incorporated by reference in its entirety) in which compounds were designed to convert into cytarabine in the liver and plasma. WO 2004/041203, herein incorporated by reference in its entirety, discloses prodrugs of gemcitabine, where some of the prodrugs are N4-carbamates. These compounds were designed to overcome the gastrointestinal toxicity of gemcitabine and were intended to provide gemcitabine by hydrolytic release in the liver and plasma after absorption of the intact prodrug from the gastrointestinal tract. Nomura et al (Bioorg Med. Chem. 2003, 11, 2453-61, herein incorporated by reference in its entirety) have described acetal derivatives of 1-(3-C-ethynyl-β-D-ribo-pentofaranosyl) cytosine which, on bioreduction, produced an intermediate that required further hydrolysis under acidic conditions to produce a cytotoxic nucleoside compound.

Cytotoxic nucleotides which may be chemotherapeutic also include, but are not limited to, pyrazolo[3,4-D]-pyrimidines, allopurinol, azathioprine, capecitabine, cytosine arabinoside, fluorouracil, mercaptopurine, 6-thioguanine, acyclovir, ara-adenosine, ribavirin, 7-deaza-adenosine, 7-deaza-guanosine, 6-aza-uracil, 6-aza-cytidine, thymidine ribonucleotide, 5-bromodeoxyuridine, 2-chloro-purine, and inosine, or combinations thereof.

Coding Sequences

In some aspects of the disclosure, the modified RNA comprises a sequence that encodes an interleukin (IL)-12 molecule. In some aspects, the IL-12 molecule comprises is IL-12, an IL-12 subunit, or a mutant IL-12 molecule that retains immunomodulatory function.

In some aspects, the IL-12 comprises an amino acid sequence at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 1.

In some aspects, the IL-12 molecule comprises IL-12a and/or IL-120 subunits. In some aspects, the IL-12a subunit comprises an amino acid sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 2.

In some aspects, the IL-120 subunit comprises an amino acid sequence at least about 70%, at least about 75%, at least about 80%, at least about 85%, at least about 90%, at least about 91%, at least about 92%, at least about 93%, at least about 94% at least about 95%, at least about 96%, at least about 97%, at least about 98%, at least about 99%, or about 100% identical to SEQ ID NO: 3.

In some aspects, the IL-12a subunit and the IL-120 subunit are linked by a linker. In some aspects, the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids. In some aspects, the linker comprises a (GS) linker. In some aspects, the GS linker has a formula of (Gly4Ser)n or S(Gly4Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100. In some aspects, the (Gly4Ser)n linker is (Gly4Ser)3 or (Gly4Ser)4.

In some aspects, the IL12 molecule comprises an amino acid sequence that is at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 4 or SEQ ID NO: 5

In some aspects, the modified mRNA comprises a nucleotide sequence that is at least about 70%, at least about 75%, at least about 80%, at least about 85%, at least about 90%, at least 91%, at least about 92%, at least about 93%, at least about 94%, at least about 95%, at least about 96%, at least about 97%, at least about 98%, at least about 99%, or about 100% identical to SEQ ID NO: 6.

In some aspects, the modified RNA comprises a nucleotide sequence that is at least about 70%, at least about 75%, at least about 80%, at least about 85%, at least about 90%, at least 91%, at least about 92%, at least about 93%, at least about 94%, at least about 95%, at least about 96%, at least about 97%, at least about 98%, at least about 99%, or about 100% identical to SEQ ID NO: 7.

In some aspects, the modified RNA comprises a nucleotide sequence that is at least about 70%, at least about 75%, at least about 80%, at least about 85%, at least about 90%, at least 91%, at least about 92%, at least about 93%, at least about 94%, at least about 95%, at least about 96%, at least about 97%, at least about 98%, at least about 99%, or about 100% identical to SEQ ID NO: 8.

In some aspects, the modified RNA comprises a nucleotide sequence that is at least about 70%, at least about 75%, at least about 80%, at least about 85%, at least about 90%, at least 91%, at least about 92%, at least about 93%, at least about 94%, at least about 95%, at least about 96%, at least about 97%, at least about 98%, at least about 99%, or about 100% identical to SEQ ID NO: 9.

In some aspects, the modified RNA comprises a nucleotide sequence or encodes for an amino acid having at least about 70%, at least about 75%, at least about 80%, at least about 85%, at least about 90%, at least 91%, at least about 92%, at least about 93%, at least about 94%, at least about 95%, at least about 96%, at least about 97%, at least about 98%, at least about 99%, or about 100% identity to a sequence in Table 2.

Additional sequences are disclosed in International Application No. WO2017201350 and WO2018/160540, which are herein incorporated by reference in their entirety.

TABLE 2

| SEQ ID NO | Description | Sequence |
|---|---|---|
| 1 | Wild-type | MCPARSLLLVATLVLLDHLSLARNLPVATPDPGMFPCLHHSQNLLRAVSN |
| | hIL-12 | MLQKARQTLEFYPCTSEEIDHEDITKDKTSTVEACLPLELTKNESCLNSR |
| | | ETSFITNGSCLASRKTSFMMALCLSSIYEDLKMYQVEFKTMNAKLLMDPK |
| | | RQIFLDQNMLAVIDELMQALNFNSETVPQKSSLEEPDFYKTKIKLCILLH |
| | | AFRIRAVTIDRVMSYLNASMCHQQLVISWFSLVFLASPLVAIWELKKDVY |
| | | VVELDWYPDAPGEMVVLTCDTPEEDGITWTLDQSSEVLGSGKTLTIQVKE |
| | | FGDAGQYTCHKGGEVLSHSLLLLHKKEDGIWSTDILKDQKEPKNKTFLRC |
| | | EAKNYSGRFTCWWLTTISTDLTFSVKSSRGSSDPQGVTCGAATLSAERVR |
| | | GDNKEYEYSVECQEDSACPAAEESLPIEVMVDAVHKLKYENYTSSFFIRD |
| | | IIKPDPPKNLQLKPLKNSRQVEVSWEYPDTWSTPHSYFSLTFCVQVQGKS |
| | | KREKKDRVFTDKTSATVICRKNASISVRAQDRYYSSSWSEWASVPCS |
| 2 | IL-12α | MCPARSLLLVATLVLLDHLSLARNLPVATPDPGMFPCLHHSQNLLRAVSN |
| | | MLQKARQTLEFYPCTSEEIDHEDITKDKTSTVEACLPLELTKNESCLNSR |
| | | ETSFITNGSCLASRKTSFMMALCLSSIYEDLKMYQVEFKTMNAKLLMDPK |
| | | RQIFLDQNMLAVIDELMQALNFNSETVPQKSSLEEPDFYKTKIKLCILLH |
| | | AFRIRAVTIDRVMSYLNAS |
| 3 | IL-12β | MCHQQLVISWFSLVFLASPLVAIWELKKDVYVVELDWYPDAPGEMVVLTC |
| | | DTPEEDGITWTLDQSSEVLGSGKTLTIQVKEFGDAGQYTCHKGGEVLSHS |
| | | LLLLHKKEDGIWSTDILKDQKEPKNKTFLRCEAKNYSGRFTCWWLTTIST |
| | | DLTFSVKSSRGSSDPQGVTCGAATLSAERVRGDNKEYEYSVECQEDSACP |
| | | AAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVIC |
| | | RKNASISVRAQDRYYSSSWSEWASVPCS |
| 4 | Modified IL- | MCHQQLVISWFSLVFLASPLVAIWELKKDVYVVELDWYPDAPGEMVVLTC |
| | 12 amino acid | DTPEEDGITWTLDQSSEVLGSGKTLTIQVKEFGDAGQYTCHKGGEVLSHS |
| | | LLLLHKKEDGIWSTDILKDQKEPKNKTFLRCEAKNYSGRFTCWWLTTIST |
| | | DLTFSVKSSRGSSDPQGVTCGAATLSAERVRGDNKEYEYSVECQEDSACP |
| | | AAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVIC |
| | | RKNASISVRAQDRYYSSSWSEWASVPCSGGGGGGSRNLPVATPDPGMFPC |
| | | LHHSQNLLRAVSNMLQKARQTLEFYPCTSEEIDHEDITKDKTSTVEACLP |
| | | LELTKNESCLNSRETSFITNGSCLASRKTSFMMALCLSSIYEDLKMYQVE |
| | | FKTMNAKLLMDPKRQIFLDQNMLAVIDELMQALNFNSETVPQKSSLEEPD |
| | | FYKTKIKLCILLHAFRIRAVTIDRVMSYLNAS |
| 5 | Modified IL- | MCHQQLVISWFSLVFLASPLVAIWELKKDVYVVELDWYPDAPGEMVVLTC |
| | 12 amino acid | DTPEEDGITWTLDQSSEVLGSGKTLTIQVKEFGDAGQYTCHKGGEVLSHS |
| | | LLLLHKKEDGIWSTDILKDQKEPKNKTFLRCEAKNYSGRFTCWWLTTIST |
| | | DLTFSVKSSRGSSDPQGVTCGAATLSAERVRGDNKEYEYSVECQEDSACP |
| | | AAEESLPIEVMVDAVHKLKYENYTSSFFIRDIIKPDPPKNLQLKPLKNSR |
| | | QVEVSWEYPDTWSTPHSYFSLTFCVQVQGKSKREKKDRVFTDKTSATVIC |
| | | RKNASISVRAQDRYYSSSWSEWASVPCSGSSGGGGSPGGGSSRNLPVATP |
| | | DPGMFPCLHHSQNLLRAVSNMLQKARQTLEFYPCTSEEIDHEDITKDKTS |
| | | TVEACLPLELTKNESCLNSRETSFITNGSCLASRKTSFMMALCLSSIYED |
| | | LKMYQVEFKTMNAKLLMDPKRQIFLDQNMLAVIDELMQALNFNSETVPQK |
| | | SSLEEPDFYKTKIKLCILLHAFRIRAVTIDRVMSYLNAS |
| 6 | Modified IL- | augugccaccagcagcuggugaucagcugguucagccugguguuccuggc |
| | 12 sequence | cagcccccugguggccaucugggagcugaagaaggacguguacguggugg |
| | | aguuggauugguaccccgacgcccccggcgagaugguggugcugaccugc |
| | | gacacccccgaggaggacggcaucaccuggacccuggaccagagcagcga |
| | | ggugcugggcagcggcaagacccugaccauccaggugaaggaguucggcg |
| | | acgccggccaguacaccugccacaagggcggcgaggugcugagccacagc |
| | | cugcugcugcugcacaagaaggaggacggcaucuggagcaccgacauccu |
| | | gaaggaccagaaggagcccaagaacaagaccuuccugagaugcgaggcca |
| | | agaacuacagcggcagauucaccugcugguggcugaccaccaucagcacc |
| | | gaccugaccuucagcgugaagagcagcagaggcagcagcgacccccaggg |
| | | cgugaccugcggcgccgccacccugagcgccgagagagugagaggcgaca |
| | | acaaggaguacgaguacagcguggagugccaggaagauagcgccugcccc |
| | | gccgccgaggagagccugcccaucgaggugaugguggacgccgugcacaa |
| | | gcugaaguacgagaacuacaccagcagcuucuucaucagagauaucauca |
| | | agcccgacccccccaagaaccugcagcugaagccccugaagaacagccgg |
| | | cagguggaggugagcugggaguaccccgacaccuggagcaccccccacag |
| | | cuacuucagccugaccuucugcgugcaggugcagggcaagagcaagagag |
| | | agaagaaagauagaguguucaccgacaagaccagcgccaccgugaucugc |
| | | agaaagaacgccagcaucagcgugagagcccaagauagauacuacagcag |
| | | cagcuggagcgagugggccagcgugcccugcagcggcggcggcggcggcg |
| | | gcagcagaaaccugcccguggccacccccgaccccggcauguuccccugc |
| | | cugcaccacagccagaaccugcugagagccgugagcaacaugcugcagaa |
| | | ggcccggcagacccuggaguucuaccccugcaccagcgaggagaucgacc |
| | | acgaagauaucaccaaagauaagaccagcaccguggaggccugccugccc |
| | | cuggagcugaccaagaacgagagcugccugaacagcagagagaccagcuu |
| | | caucaccaacggcagcugccuggccagcagaaagaccagcuucaugaugg |
| | | cccugugccugagcagcaucuacgaggaccugaagauguaccagguggag |
| | | uucaagaccaugaacgccaagcugcugauggaccccaagcggcagaucuu |
| | | ccuggaccagaacaugcuggccgugaucgacgagcugaugcaggcccuga |
| | | acuucaacagcgagaccgugccccagaagagcagccuggaggagcccgac |
| | | uucuacaagaccaagaucaagcugugcauccugcugcacgccuucagaau |
| | | cagagccgugaccaucgacagagugaugagcuaccugaacgccagc |
| 7 | Modified IL- | augugucaccagcaguuggucaucucuugguuuucccugguuuuucuggc |
| | 12 sequence | aucuccccucguggccauaugggaacugaagaaagauguuuaugucguag |
| | | aauuggauugguauccggaugccccuggagaaauggugguccucaccugu |
| | | gacaccccugaagaagaugguaucaccuggaccuuggaccagagcaguga |
| | | ggucuuaggcucuggcaaaacccugaccauccaagucaaagaguuuggag |
| | | augcuggccaguacaccugucacaaaggaggcgagguucuaagccauucg |
| | | cuccugcugcuucacaaaaaggaagauggaauuugguccacugauauuuu |
| | | aaaggaccagaaagaacccaaaaauaagaccuuucuaagaugcgaggcca |
| | | agaauuauucuggacguuucaccugcugguggcugacgacaaucaguacu |
| | | gauuugacauucagugucaaaagcagcagagggucuucugacccccaagg |
| | | ggugacgugcggagcugcuacacucucugcagagagagucagaggggaca |
| | | acaaggaguaugaguacucaguggagugccaggaggacagugccugccca |
| | | gcugcugaggagagucugcccauugaggucaugguggaugccguucacaa |
| | | gcucaaguaugaaaacuacaccagcagcuucuucaucagggacaucauca |
| | | aaccugacccacccaagaacuugcagcugaagccauuaaagaauucucgg |
| | | cagguggaggucagcugggaguacccugacaccuggaguacuccacauuc |
| | | cuacuucucccugacauucugcguucagguccagggcaagagcaagagag |
| | | aaaagaaagauagagucuucacggacaagaccucagccacggucaucugc |
| | | cgcaaaaaugccagcauuagcgugcgggcccaggaccgcuacuauagcuc |
| | | aucuuggagcgaaugggcaucugugcccugcaguggcucuagcggagggg |
| | | gaggcucuccuggcgggggaucuagcagaaaccuccccguggccacucca |
| | | gacccaggaauguucccaugccuucaccacucccaaaaccugcugagggc |
| | | cgucagcaacaugcuccagaaggccagacaaacucuagaauuuuacccuu |
| | | gcacuucugaggaaauugaucaugaagauaucacaaaagauaaaaccagc |
| | | acaguggaggccuguuuaccauuggaauuaaccaagaaugagaguugccu |
| | | aaauuccagagagaccucuuucauaacuaaugggaguugccuggccucca |
| | | gaaagaccucuuuuaugauggcccugugccuuaguaguauuuaugaagac |
| | | uugaagauguaccagguggaguucaagaccaugaaugcaaagcuucugau |
| | | ggauccuaagaggcagaucuuucuagaucaaaacaugcuggcaguuauug |
| | | augagcugaugcaggcccugaauuucaacagugagacugugccacaaaaa |
| | | uccucccuugaagaaccggauuuuuauaaaacuaaaaucaagcucugcau |
| | | acuucuucaugcuuucagaauucgggcagugacuauugauagagugauga |
| | | gcuaucugaaugcuuccugauga |
| 8 | Modified IL- | ggggaaataagagagaaaagaagagtaagaagaaatataagagccaccat |
| | 12 nucleic | gtgccaccagcagctggtgatcagctggttcagcctggtgttcctggcca |
| | acid sequence | gccccctggtggccatctgggagctgaagaaggacgtgtacgtggtggag |
| | | ttggattggtaccccgacgc |
| | | ccccggcgagatggtggtgctgacctgcgacacccccgaggaggacggca |
| | | tcacctggaccctggaccagagcagcgaggtgctgggcagcggcaagacc |
| | | ctgaccatccaggtgaaggagttcggcgacgccggccagtacacctgcca |
| | | caagggcggcgaggtgctgagccacagcctgctgctgctgcacaagaagg |
| | | aggacggcatctggagcaccgacatcctgaaggaccagaaggagcccaag |
| | | aacaagaccttcctgagatgcgaggccaagaactacagcggcagattcac |
| | | ctgctggtggctgaccaccatcagcaccgacctgaccttcagcgtgaaga |
| | | gcagcagaggcagcagcgacccccagggcgtgacctgcggcgccgccacc |
| | | ctgagcgccgagagagtgagaggcgacaacaaggagtacgagtacagcgt |
| | | ggagtgccaggaagatagcgcctgccccgccgccgaggagagcctgccca |
| | | tcgaggtgatggtggacgccgtgcacaagctgaagtacgagaactacacc |
| | | agcagcttcttcatcagagatatcatcaagcccgacccccccaagaacct |
| | | gcagctgaagcccctgaagaacagccggcaggtggaggtgagctgggagt |
| | | accccgacacctggagcaccccccacagctacttcagcctgaccttctgc |
| | | gtgcaggtgcagggcaagagcaagagagagaagaaagatagagtgttcac |
| | | cgacaagaccagcgccaccgtgatctgcagaaagaacgccagcatcagcg |
| | | tgagagcccaagatagatactacagcagcagctggagcgagtgggccagc |
| | | gtgccctgcagcggcggcggcggcggcggcagcagaaacctgcccgtggc |
| | | cacccccgaccccggcatgttcccctgcctgcaccacagccagaacctgc |
| | | tgagagccgtgagcaacatgctgcagaaggcccggcagaccctggagttc |
| | | tacccctgcaccagcgaggagatcgaccacgaagatatcaccaaagataa |
| | | gaccagcaccgtggaggcctgcctgcccctggagctgaccaagaacgaga |
| | | gctgcctgaacagcagagagaccagcttcatcaccaacggcagctgcctg |
| | | gccagcagaaagaccagcttcatgatggccctgtgcctgagcagcatcta |
| | | cgaggacctgaagatgtaccaggtggagttcaagaccatgaacgccaagc |
| | | tgctgatggaccccaagcggcagatcttcctggaccagaacatgctggcc |
| | | gtgatcgacgagctgatgcaggccctgaacttcaacagcgagaccgtgcc |
| | | ccagaagagcagcctggaggagcccgacttctacaagaccaagatcaagc |
| | | tgtgcatcctgctgcacgccttcagaatcagagccgtgaccatcgacaga |
| | | gtgatgagctacctgaacgccagctgataataggctggagcctcggtggc |
| | | catgcttcttgccccttgggcctccccccagcccctcctccccttcctgc |
| | | acccgtaccccccaaacaccattgtcacactccagtggtctttgaataaa |
| | | gtctgagtgggcggcaaaaaaaaaaaaaaaaaaaaaaaaaaaaaaaaaaa |
| | | aaaaaaaaaaaaaaaaaaaaaaaaaaaaaaaaaaaaaaaaaaaaaaaaaa |
| | | aaaaaaaaaaaaaaatctag |
| 9 | Modified IL- | augugucaccagcagcuggugaucucaugguucucccugguauuucuggc |
| | 12 nucleic | aucuccucuugucgcaaucugggaacugaagaaagacguguaugucguug |
| | acid sequence | agcucgacugguauccggaugcgccuggcgagaugguggugcugaccugu |
| | | gacaccccagaggaggaugggaucacuuggacccuugaucaauccuccga |
| | | agugcucgggucuggcaagacucugaccauacaagugaaagaguuuggcg |
| | | augccgggcaguacacuugccauaagggcggagaaguucugucccacuca |
| | | cugcugcugcugcacaagaaagaggacggaauuuggaguaccgauauccu |
| | | gaaagaucagaaagagcccaagaacaaaaccuucuugcggugcgaagcca |
| | | agaacuacucagggagauuuacuuguugguggcugacgacgaucagcacc |
| | | gaucugacuuucuccgugaaaucaaguaggggaucaucugacccucaagg |
| | | agucacauguggagcggcuacucugagcgcugaacgcguaagaggggaca |
| | | auaaggaguacgaguauagcguugagugccaagaggauagcgcaugcccc |
| | | gccgccgaagaaucauugcccauugaagugaugguggaugcuguacacaa |
| | | gcugaaguaugagaacuacacaagcuccuucuucauccgugacaucauca |
| | | aaccagauccuccuaagaaccuccagcuuaaaccucugaagaacucuaga |
| | | cagguggaagugucuugggaguaucccgacaccuggucuacaccacauuc |
| | | cuacuucagucucacauucugcguucagguacagggcaaguccaaaaggg |
| | | agaagaaggaucgggucuuuacagauaaaacaagugccaccguuauaugc |
| | | cggaagaaugccucuauuucugugcgugcgcaggacagauacuauagcag |
| | | cucuuggagugaaugggccagugucccauguucagggucauccgguggug |
| | | gcggcagccccggaggcgguagcuccagaaaucucccuguggcuacaccu |
| | | gauccaggcauguuucccuguuugcaccauagccaaaaccuccugagagc |
| | | agucagcaacaugcuccagaaagcuagacaaacacuggaauucuacccau |
| | | gcaccuccgaggaaauagaucacgaggauaucacuaaggacaaaacaagc |
| | | acugucgaagcaugccuucccuuggaacugacaaagaacgagaguugccu |
| | | uaauucaagagaaacaucuuucauuacaaacgguagcugcuuggcaagca |
| | | gaaaaacaucuuuuaugauggcccuuugucugagcaguauuuaugaggau |
| | | cucaaaauguaccagguggaguuuaagaccaugaaugccaagcugcugau |
| | | ggacccaaagagacagauuuuccucgaucagaauaugcuggcugugauug |
| | | augaacugaugcaggccuugaauuucaacagcgaaaccguuccccagaaa |
| | | agcagucuugaagaaccugacuuuuauaagaccaagaucaaacuguguau |
| | | ucuccugcaugccuuuagaaucagagcagucacuauagauagagugaugu |
| | | ccuaccugaaugcuuccugauga |

In some aspects, the modified RNA comprises one or more gene(s) of experimental or therapeutic interest. In some aspects, the gene(s) of experimental or therapeutic interest encode cytokines, chemokines, or growth factors other than IL-12. Cytokines are known in the art, and the term itself refers to a generalized grouping of small proteins that are secreted by certain cells within the immune system and have an effect on other cells. Cytokines are known to enhance the cellular immune response and, as used herein, can include, but are not limited to, TNFα, IFN-γ, IFN-α, TGFβ, IL-1, IL-2, 11-4, IL-10, IL-13, IL-17, IL-18, and chemokines. Chemokines are useful for studies investigating response to infection, immune responses, inflammation, trauma, sepsis, cancer, and reproduction, among other applications. Chemokines are known in the art, and are a type of cytokine that induce chemotaxis in nearby responsive cells, typically of white blood cells, to sites of infection. Non-limiting examples of chemokines include, CCL14, CCL19, CCL20, CCL21, CCL25, CCL27, CXCL12, CXCL13, CXCL-8, CCL2, CCL3, CCL4, CCL5, CCL11, AND CXCL10. Growth factors are known in the art and the term itself is sometimes interchangeable with the term cytokines. As used herein, the term “growth factors” refers to a naturally occurring substance capable of signaling between cells and stimulating cellular growth. While cytokines may be growth factors, certain types of cytokines may also have an inhibitory effect on cell growth, thus differentiating the two terms. Non-limiting examples of growth factors include Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (m-CSF), Granulocyte colony-stimulating factor (G-CSF), Granulocyte macrophage colony-stimulating factor (GM-CSF), Epidermal growth factor (EFG), Ephrin A1, Ephrin A2, Ephrin A3, Ephrin A4, Ephrin A5, Ephrin B1, Ephrin B2, Ephrin B3, Erythropoietin (EPO), Fibroblast frowth factor-1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15(FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Fetal Bovine Somatotrophin (FBS), Glial cell line-derived neurotrophic factor (GDNF), Neurturin, Peresphin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), Myostatin (GDF-8), Neuregulin 1 (NRG1), Neuregulin 2 (NRG2), Neuregulin 3 (NRG3), Neuregulin 4 (NRG 4), Brain-derived neurotrophic factor (BDNF), Nerve growth factor (NGF), Neurotrophin-3 (NT-3), Neurotrophin-4 (NT-4), Placental growth factor (TCGF), Thrombopoietin (TPO), Transforming growth factor alpha (TGF-α), Transforming growth factor beta (TGF-β), Tumor necrosis factor-alpha (TNF-α), and Vascular endothelial growth factor (VEGF).

Pharmaceutical Compositions

In some aspects, the disclosure relates to a pharmaceutical composition comprising the lipid nanoparticle described herein. In some aspects of the disclosure, the pharmaceutical composite on further comprises a pharmaceutically acceptable carrier (excipient) to form a pharmaceutical composition for use in treating a target disease. “Acceptable”, as used herein, means that the carrier must be compatible with the active ingredient of the composition and not deleterious to the subject to be treated. In some aspects, the carrier is capable of stabilizing the active ingredient. Pharmaceutically acceptable excipients (carriers) include buffers, which are well known in the art. See, e.g., Remington: The Science and Practice of Pharmacy 20th Ed. (2000) Lippincott Williams and Wilkoins, Ed. K. E. Hoover.

The pharmaceutical compositions to be used for in vivo administration must be sterile. This is readily accomplished by, for example, filtration through sterile filtration membranes. The lipid nanoparticles may be placed into a container having a sterile access port, for example, an intravenous solution bag or vial having a stopper pierceable by a hypodermic injection needle.

In some aspects of the disclosure, the pharmaceutical composition can be formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration. In some aspects of the disclosure, the pharmaceutical composition can be formulated for intratumoral injection. Intratumoral injection, as used herein, refers to direct injections into the tumor. A high concentration of composition can be achieved in situ, while using small amounts of drugs. Local delivery of immunotherapies allows multiple combination therapies, while preventing significant system exposure and off-target toxicities.

In some aspects of the disclosure, the pharmaceutical composition can be formulated for intramuscular injection, intravenous injection, or subcutaneous injection.

In some aspects of the disclosure, the pharmaceutical composition comprises pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover. Acceptable carriers and excipients, or stabilizers are nontoxic to recipients at the dosages and concentrations used, and comprises buffers such as phosphate, citrate, and other organic acds; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

In some aspects, the pharmaceutical composition described herein comprises lipid nanoparticles which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545, which are hereby incorporated by reference in their entirety. Liposomes with enhanced circulation time are disclosed in U.S. Pat. No. 5,013,556, which is hereby incorporated by reference in its entirety. In some aspects, liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some aspects of the disclosure, the pharmaceutical composition is formulated in sustained-release format. Suitable examples of sustained-release preparations include semipermeable matrices of solid hydrophobic polymers containing the lipid nanoparticles which matrices are in the form of shaped articles, e.g., films or microcapsules. Examples of sustained-release matrices include, but are not limited to, polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPROM DEPOT™ (injectable microspheres composed of lactic acid-glycolic acid copolymer and leuprolide acetate), sucrose acetate isobutyrate, and poly-D-(−)-3-hydroxybutyric acid.

In some aspects, suitable surface-active agents include, but are not limited to, non-ionic agents, such as polyoxyethylenesorbitans (e.g., TWEEN™ 20, 40, 60, 80 or 85) and other sorbitans (e.g., SPAN™ 20, 30, 60, 80, or 85). In some aspects, compositions with a surface-active agent comprise between 0.05 and 5% surface-active agent. In some aspects the composition comprises 0.1 and 2.5%. It will be appreciated that other ingredients may be added, for example mannitol or other pharmaceutically acceptable vehicles, if necessary.

In some aspects, the pharmaceutical composition is in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g, water, to form a solid preformulation composition containing a homogenous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

Suitable emulsions may be prepared using commercially available fat emulsions, such as INTRALIPID™, LIPOSYN™, INFONUTROL™, LIPOFUNDIN™, and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil, or almond oil) and an emulsion formed upon mixing with a phospholipid (e.g., egg phospholipids, soybean phospholipids, or soybean lecithin) and water. It will be appreciated that other ingredients may be added, for example glycerol or glucose, to adjust tonicity of the emulsion. Suitable emulsions will typically contain up to 20% oil, for example, between 5 and 20%. The fat emulsion can comprise fat droplets having a suitable size and can have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some aspects, the composition is administered by the oral or nasal respiratory route for local or systemic effect.

Compositions in pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension, or powder compositions may be administered from devices which deliver the formulation in an appropriate manner.

Therapeutic Applications

In some aspects of the disclosure, the lipid nanoparticles or pharmaceutical compositions described herein are used to treat cancer.

In some aspects, an effective amount of any of the lipid nanoparticles or pharmaceutical compositions described herein is administered to a subject in need thereof via a suitable route, such as intratumoral administration, intravenous administration (e.g, as a bolus or by continuous infusion over a period of time), by intramuscular, intraperitoneal, intracerebospinal, subcutaneous, intra-articular, intrasynovial, intrathecal, oral, inhalation, or topical routes. Commercially available nebulizers for liquid formulations, including jet nebulizers and ultrasonic nebulizers are useful for administration. Liquid formulations can be nebulized and lyophilized powder can be nebulized after reconstitution. In some aspects, the pharmaceutical composition described herein is aerolized using a fluorocarbon formulation and a metered dose inhaler, or inhaled as a lyophilized and milled powder. In some aspects, the pharmaceutical composition described herein is formulated for intratumoral injection. In some aspects, the pharmaceutical composition described herein is administered to a subject via a local route, for example, injected to a local site such as a tumor site or an infectious site. In some aspects, the subject is a human.

As used herein, “an effective amount” refers to the amount of each active agent required to confer therapeutic effect on the subject, either alone or in combination with one or more other active agents. In some aspects, the therapeutic effects is reduced tumor burden, reduction of cancer cells, or increased immune activity. Determination of whether a lipid nanoparticle achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of the concurrent therapy (if any), the specific route of administration and like factors within the knowledge of expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of a lipid nanoparticle may be appropriate. Various formulations and devices for achieving sustained release are known in the art.

In some aspects of the disclosure, the treatment is a single injection of the lipid nanoparticle or pharmaceutical composition disclosed herein. In some aspects, the single injection is administered intratumorally to the subject in need thereof.

In some aspects of the disclosure, dosages for a lipid nanoparticle or pharmaceutical composition described herein may be determined empirically in individuals who have been given one or more administration(s) of lipid nanoparticle. Individuals are given incremental dosages of the lipid nanoparticle or pharmaceutical composition described herein. To assess efficacy of the lipid nanoparticle or pharmaceutical composition herein, an indicator of disease/disorder can be followed. For repeated administrations over several days or longer, depending on the condition, the treatment is sustained until a desired suppression of symptoms occurs or until sufficient therapeutic levels are achieved to alleviate a target disease or disorder, or symptom thereof.

In some aspects of the disclosure, dosing frequency is once every week, every 2 weeks, every 3 weeks, every 4 weeks, every 5 weeks, every 6 weeks, every 7 weeks, every 8 weeks, every 9 weeks, or every 10 weeks; or once a month, every 2 months, or every 3 months, or longer. The progress of this therapy is easily monitored by conventional techniques and assays. The dosing regimen of the lipid nanoparticle used can vary over time.

In some aspects of the disclosure, the method comprises administering to a subject in need thereof one or multiple doses of a lipid nanoparticle or pharmaceutical composition described herein.

The appropriate dosage of the lipid nanoparticle described herein will depend on the specific lipid nanoparticle, the type and severity of the disease/disorder, the if the lipid nanoparticle is administered for preventive or therapeutic purposes, previous therapy, the subject's clinical history and response to the lipid nanoparticle, and the discretion of the attending physician. A clinician may administer a lipid nanoparticle or pharmaceutical composition disclosed herein until a dosage is reached that achieves the desired result. In some aspects, the desired result is a decrease in tumor burden, a decrease in cancer cells, or increased immune activity. Methods of determining whether a dosage resulted in the desired result would be evident to one of skill in the art. Administration of one or more lipid nanoparticles or pharmaceutical compositions described herein can be continuous or intermittent, depending, for example, upon the recipient's physiological condition, whether the purpose of the administration is therapeutic or prophylactic, and other factors known to skilled practitioners. The administration of the lipid nanoparticle or pharmaceutical composition described herein may be essentially continuous over a preselected period of time or may be in a series of spaced doses, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

As used herein, alleviating a target disease/disorder includes delaying the development or progression of the disease, or reducing disease severity. Alleviating the disease does not necessarily require curative results. As used herein, “delaying” the development of a target disease or disorder means to defer, hinder, slow, retard, stabilize, and/or postpone progression of the disease. This delay can be for varying lengths of time, depending on the history of the disease and/or subject being treated. A method that delays or alleviates the development of a disease, or delays the onset of the disease, is a method that reduces probability of developing one or more symptoms of the disease in a given time frame and/or reduces the extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be unpredictable. As used herein, development or progression refers to the biological course of symptoms. Development includes occurrence, recurrence, and onset. As used herein, onset or occurrence of a target disease or disorder includes initial onset and/or recurrence.

In some aspects, a lipid nanoparticle or pharmaceutical composition described herein is administered to a subject in need thereof at an amount sufficient to reduce tumor burden or cancer cell growth in vivo by at least 5%, at least 10%, at least 20%, at least 30%, at least 40%, at least 50%, at least 60%, at least 70%, at least 80%, at least 90%, or greater. In some aspects, the lipid nanoparticle or pharmaceutical composition described herein is administered in an amount effective in increasing immune activity by at least 5%, at least 10%, at least 20%, at least 30%, at least 40%, at least 50%, at least 60%, at least 70%, at least 80%, at least 90%, or greater.

In some aspects, the subject is a human, farm animal, sport animal, pet, primate, horse, dog, cat, mice, or rat. In some aspects, the subject is a human. In some aspects, the lipid nanoparticle or pharmaceutical composition described herein enhances immune activity, such as T cell activity, in the subject.

In some aspects, the subject is a human having, suspected of having, or at risk for a cancer. In some aspects the cancer is selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and various types of head and neck cancer, including squamous cell head and neck cancer. In some aspects, the cancer can be melanoma, lung cancer, colorectal cancer, renal-cell cancer, urothelial carcinoma, or Hodgkin's lymphoma.

A subject having a target disease or disorder can be identified by routine medical examination, e.g., laboratory tests, organ functional tests, CT scans, or ultrasounds. A subject suspected of having a target disease or disorder might show one or more symptoms of the disease or disorder. A subject at risk for the disease or disorder can be a subject having one or more of the risk factors associated with that disease or disorder. A subject at risk for a disease or disorder can also be identified by routine medical practices.

In some aspects, the lipid nanoparticle or pharmaceutical composition described herein is co-administered with at least one additional suitable therapeutic agent. In some aspects the at least one additional suitable therapeutic agent is an anti-cancer agent, an anti-viral agent, an anti-bacterial agent, or other agents that serve to enhance and/or complement the immunostimulatory effect of the lipid nanoparticle described herein. In some aspects, the lipid nanoparticle or pharmaceutical composition described herein and the at least one additional therapeutic agent are administered to the subject in a sequential manner, i.e, each therapeutic agent is administered at a different time. In some aspects, the lipid nanoparticle or pharmaceutical composition described herein and the at least one additional therapeutic agent are administered to the subject in a substantially simultaneous manner.

In some aspects, the lipid nanoparticle or pharmaceutical composition described herein may be combined with the administration of other biologically active ingredients (e.g, a different anti-cancer therapy), non-drug therapies (e.g., surgery), or combinations thereof.

It will be appreciated by one of skill in the art that any combination of the lipid nanoparticle or pharmaceutical composition described herein and another anti-cancer agent (e.g., a chemotherapeutic agent) may be used in any sequence for treating a cancer. The combinations described herein may be selected on the basis of a number of factors, which include but are not limited to, the effectiveness or reducing tumor formation or tumor growth, reducing cancer cells, increasing immune activity, and/or alleviating at least one symptom associated with the cancer, or the effectiveness for mitigating the side effects of another agent of the combination. For example, a combined therapy described herein may reduce any of the side effects associated with each individual members of the combination, for example, a side effect associated with the anti-cancer agent.

In some aspects, the other anti-cancer therapeutic agent is a chemotherapy, a radiation therapy, a surgical therapy, an immunotherapy, or combinations thereof. In some aspects, the chemotherapeutic agent is carboplatin, cisplatin, docetaxel, gemcitabine, nab-paclitexal, pemetrexed, vinorelbine, or combinations thereof. In some aspects, the radiation therapy is ionizing radiation, gamma-radiation, neutron beam radiotherapy, electron beam radiotherapy, proton therapy, brachytherapy, systemic radioactive isotopes, radiosensitizers, or combinations thereof. In some aspects, the surgical therapy is a curative surgery (e.g., tumor removal surgery), a preventative surgery, a laparoscopic surgery, a laser surgery, or combinations thereof. In some aspects, the immunotherapy is adoptive cell transfer, therapeutic cancer vaccines, or combinations thereof.

In some aspects, the chemotherapeutic agent is platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capacitabine, Gemcitabine, hydroxyurea, 5-Fluorouracil (5FU), and relatives); Alkylating agents, such as Nitrogen mustards (e.g., Cyclophosphamide, Melphalan, Chlorambucil, mechlorethamine, Ifosfamide, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemalamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); Ultraviolet light; and combinations thereof.

In some aspects, the other anti-cancer therapeutic agent is an antibody. Antibodies (preferably monoclonal antibodies) achieve their therapeutic effect against cancer cells through various mechanisms. They can have direct effects in producing apoptosis or programmed cell death. They can block components of signal transduction pathways such as e.g. growth factor receptors, effectively arresting proliferation of tumor cells. In cells that express monoclonal antibodies, they can bring about anti-idiotype antibody formation. Indirect effects include recruiting cells that have cytotoxicity, such as monocytes and macrophages. This type of antibody-mediated cell kill is called antibody-dependent cell mediated cytotoxicity (ADCC). Antibodies also bind complement, leading to direct cell toxicity, known as complement dependent cytotoxicity (CDC). Combining surgical methods with immunotherapeutic drugs or methods is an successful approach, as e.g. demonstrated in Gadri et al. 2009: Synergistic effect of dendritic cell vaccination and anti-CD20 antibody treatment in the therapy of murine lymphoma. J Immunother. 32(4): 333-40. The following list provides some non-limiting examples of anti-cancer antibodies and potential antibody targets (in brackets) which can be used in combination with the present invention: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Bavituximab (phosphatidylserine), Bectumomab (CD22), Belimumab (BAFF), Bevacizumab (VEGF-A), Bivatuzumab mertansine (CD44 v6), Blinatumomab (CD19), Brentuximab vedotin (CD30 TNFRSF8), Cantuzumab mertansin (mucin CanAg), Cantuzumab ravtansine (MUC1), Capromab pendetide (prostatic carcinoma cells), Carlumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R²), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-I), Gemtuzumab ozogamicin (CD33), Gevokizumab (IL-1β), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD152), Iratumumab (CD30), Labetuzumab (CEA), Lexatumumab (TRAIL-R2), Libivirumab (hepatitis B surface antigen), Lintuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL-5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Narnatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL-6), Tabalumab (BAFF), Tacatuzumab tetraxetan (alpha-fetoprotein), Taplitumomab paptox (CD19), Tenatumomab (tenascin C), Teprotumumab (CD221), Ticilimumab (CTLA-4), Tigatuzumab (TRAIL-R2), TNX-650 (IL-13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1BB), Volociximab (integrin α5 β1), Votumumab (tumor antigen CTAA16.88), Zalutumumab (EGFR), Zanolimumab (CD4).

In some aspects, the other anti-cancer therapeutic agent is a cytokine, chemokine, costimulatory molecule, fusion protein, or combinations thereof. Examples of chemokines include, but are not limited to, CCR7 and its ligands CCL19 and CCL21, furthermore CCL2, CCL3, CCL5, and CCL16. Other examples are CXCR4, CXCR7 and CXCL12. Furthermore, costimulatory or regulatory molecules such as e.g. B7 ligands (B7.1 and B7.2) are useful. Also useful are other cytokines such as e.g. interleukins especially (e.g. IL-1 to IL17), interferons (e.g. IFNalpha1 to IFNalpha8, IFNalpha10, IFNalpha13, IFNalpha14, IFNalpha16, IFNalpha17, IFNalpha21, IFNbeta1, IFNW, IFNE1 and IFNK), hematopoietic factors, TGFs (e.g. TGF-α, TGF-β, and other members of the TGF family), finally members of the tumor necrosis factor family of receptors and their ligands as well as other stimulatory molecules, comprising but not limited to 41BB, 41BB-L, CD137, CD137L, CTLA-4GITR, GITRL, Fas, Fas-L, TNFR1, TRAIL-R1, TRAIL-R2, p75NGF-R, DR6, LT.beta.R, RANK, EDAR1, XEDAR, Fn114, Troy/Trade, TAJ, TNFRII, HVEM, CD27, CD30, CD40, 4-1BB, OX40, GITR, GITRL, TACI, BAFF-R, BCMA, RELT, and CD95 (Fas/APO-1), glucocorticoid-induced TNFR-related protein, TNF receptor-related apoptosis-mediating protein (TRAMP) and death receptor-6 (DR6). Especially CD40/CD40L and OX40/OX40L are important targets for combined immunotherapy because of their direct impact on T cell survival and proliferation. For a review see Lechner et al. 2011: Chemokines, costimulatory molecules and fusion proteins for the immunotherapy of solid tumors. Immunotherapy 3 (11), 1317-1340.

In some aspects, the other anti-cancer therapeutic is a bacterial treatment. Researchers have been using anaerobic bacteria, such as Clostridium novyi, to consume the interior of oxygen-poor tumours. These should then die when they come in contact with the tumour's oxygenated sides, meaning they would be harmless to the rest of the body. Another strategy is to use anaerobic bacteria that have been transformed with an enzyme that can convert a non-toxic prodrug into a toxic drug. With the proliferation of the bacteria in the necrotic and hypoxic areas of the tumour, the enzyme is expressed solely in the tumour. Thus, a systemically applied prodrug is metabolised to the toxic drug only in the tumour. This has been demonstrated to be effective with the nonpathogenic anaerobe Clostridium sporogenes.

In some aspects, the other anti-cancer therapeutic agent is a kinase inhibitor. The growth and survival of cancer cells is closely interlocked with the deregulation of kinase activity. To restore normal kinase activity and therefor reduce tumor growth a broad range of inhibitors is in used. The group of targeted kinases comprises receptor tyrosine kinases e.g. BCR-ABL, B-Raf, EGFR, HER-2/ErbB2, IGF-IR, PDGFR-α, PDGFR-β, c-Kit, Flt-4, Flt3, FGFR1, FGFR3, FGFR4, CSF1R, c-Met, RON, c-Ret, ALK, cytoplasmic tyrosine kinases e.g. c-SRC, c-YES, Abl, JAK-2, serine/threonine kinases e.g. ATM, Aurora A & B, CDKs, mTOR, PKCi, PLKs, b-Raf, S6K, STK11/LKB1 and lipid kinases e.g. PI3K, SK1. Small molecule kinase inhibitors are e.g. PHA-739358, Nilotinib, Dasatinib, and PD166326, NSC 743411, Lapatinib (GW-572016), Canertinib (CI-1033), Semaxinib (SU5416), Vatalanib (PTK787/ZK222584), Sutent (SU11248), Sorafenib (BAY 43-9006) and Leflunomide (SU101). For more information see e.g. Zhang et al. 2009: Targeting cancer with small molecule kinase inhibitors. Nature Reviews Cancer 9, 28-39.

In some aspects, the other anti-cancer therapeutic agent is a toll-like receptor. The members of the Toll-like receptor (TLRs) family are an important link between innate and adaptive immunity and the effect of many adjuvants rely on the activation of TLRs. A large number of established vaccines against cancer incorporate ligands for TLRs for boosting vaccine responses. Besides TLR2, TLR3, TLR4 especially TLR7 and TLR8 have been examined for cancer therapy in passive immunotherapy approaches. The closely related TLR7 and TLR8 contribute to antitumor responses by affecting immune cells, tumor cells, and the tumor microenvironment and may be activated by nucleoside analogue structures. All TLR's have been used as stand-alone immunotherapeutics or cancer vaccine adjuvants and may be synergistically combined with the formulations and methods of the present invention. For more information see van Duin et al. 2005: Triggering TLR signaling in vaccination. Trends in Immunology, 27(1):49-55.

In some aspects, the other anti-cancer therapeutic agent is an angiogenesis inhibitor. Angiogenesis inhibitors prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or angiogenesis inhibitors which may be combined with the present invention are soluble VEGF (VEGF isoforms VEGF121 and VEGF165, receptors VEGFR1, VEGFR2 and co-receptors Neuropilin-1 and Neuropilin-2) 1 and NRP-1, angiopoietin 2, TSP-1 and TSP-2, angiostatin and related molecules, endostatin, vasostatin, calreticulin, platelet factor-4, TIMP and CDAI, Meth-1 and Meth-2, IFN-α, -β and -γ, CXCL10, IL-4, -12 and -18, prothrombin (kringle domain-2), antithrombin III fragment, prolactin, VEGI, SPARC, osteopontin, maspin, canstatin, proliferin-related protein, restin and drugs like e.g. bevacizumab, itraconazole, carboxyamidotriazole, TNP-470, CM101, IFN-α, platelet factor-4, suramin, SU5416, thrombospondin, VEGFR antagonists, angiostatic steroids+heparin, cartilage-derived angiogenesis Inhibitory factor, matrix metalloproteinase inhibitors, 2-methoxyestradiol, tecogalan, tetrathiomolybdate, thalidomide, thrombospondin, prolactina V133 inhibitors, linomide, tasquinimod, For review see Schoenfeld and Dranoff 2011: Anti-angiogenesis immunotherapy. Hum Vaccin. (9):976-81.

In some aspects, the other anti-cancer therapeutic agent is a virus-based vaccine. There are a number of virus-based cancer vaccines available or under development which can be used in a combined therapeutic approach together with the formulations of the present disclosure. One advantage of the use of such viral vectors is their intrinsic ability to initiate immune responses, with inflammatory reactions occurring as a result of the viral infection creating the danger signal necessary for immune activation. An ideal viral vector should be safe and should not introduce an anti-vector immune response to allow for boosting antitumour specific responses. Recombinant viruses such as vaccinia viruses, herpes simplex viruses, adenoviruses, adeno-associated viruses, retroviruses and avipox viruses have been used in animal tumour models and based on their encouraging results, human clinical trials have been initiated. Especially important virus-based vaccines are virus-like particles (VLPs), small particles that contain certain proteins from the outer coat of a virus. Virus-like particles do not contain any genetic material from the virus and cannot cause an infection but they can be constructed to present tumor antigens on their coat. VLPs can be derived from various viruses such as e.g. the hepatitis B virus or other virus families including Parvoviridae (e.g. adeno-associated virus), Retroviridae (e.g. HIV), and Flaviviridae (e.g. Hepatitis C virus). For a general review see Sorensen and Thompsen 2007: Virus-based immunotherapy of cancer: what do we know and where are we going? APMIS 115(11):1177-93; virus-like particles against cancer are reviewed in Buonaguro et al. 2011: Developments in virus-like particle-based vaccines for infectious diseases and cancer. Expert Rev Vaccines 10(11):1569-83; and in Guillén et al. 2010: Virus-like particles as vaccine antigens and adjuvants: application to chronic disease, cancer immunotherapy and infectious disease preventive strategies. Procedia in Vaccinology 2 (2), 128-133.

In some aspects, the other anti-cancer therapeutic agent is a peptide-based target therapy. Peptides can bind to cell surface receptors or affected extracellular matrix surrounding the tumor. Radionuclides which are attached to these peptides (e.g. RGDs) eventually kill the cancer cell if the nuclide decays in the vicinity of the cell. Especially oligo- or multimers of these binding motifs are of great interest, since this can lead to enhanced tumor specificity and avidity. For non-limiting examples see Yamada 2011: Peptide-based cancer vaccine therapy for prostate cancer, bladder cancer, and malignant glioma. Nihon Rinsho 69(9): 1657-61.

Kits for Use in Therapy

The present disclosure also provides kits for use in immunotherapy against cancer (e.g., melanoma, lung cancer, colorectal cancer, or renal-cell cancer), and/or treating or reducing the risk for cancer. In some aspects, the kit includes one or more containers comprising a lipid nanoparticle or pharmaceutical composition described herein.

In some aspects, the kit comprises instructions for use in accordance with any of the methods described herein. For example, the included instructions can comprise a description of administration of the pharmaceutical composition described herein to treat, delay the onset, or alleviate a target disease. In some aspects, the instructions comprise a description of administering the lipid nanoparticle or pharmaceutical composition described herein to a subject at risk of the target disease/disorder.

In some aspects, the instructions comprise dosage information, dosing schedule, and route of administration. In some aspects, the containers are unit doses, bulk packages (e.g., multi-dose packages) or sub-unit doses. In some aspects, the instructions are written instructions on a label or package insert (e.g., a paper sheet included in the kit). In some aspects, the instructions are machine-readable instructions (e.g., instructions carried on a magnetic or optical storage disk).

In some aspects, the label or package insert indicates that the lipid nanoparticle or pharmaceutical composition disclosed herein is used for treating, delaying the onset, and/or alleviating a disease or disorder associated with cancer, such as those described herein. Instructions may be provided for practicing any of the methods described herein.

In some aspects, the kits described herein are in suitable packaging. In some aspects, suitable packing comprises vials, bottles, jars, flexible packaging (e.g., seal Mylar or plastic bags), or combinations thereof. In some aspects, the packaging comprises packages for use in combination with a specific device such as an inhaler, nasal administration device (e.g., an atomizer), or an infusion device such as a minipump. In some aspects, the kit comprises a sterile access port (for example the container may be an intravenous solution bag or a vial having a stopper pierceable by a hypodermic injection needle). In some aspects, the container may also have a sterile access port (for example the container may be an intravenous solution bag or a vial having a stopper pierceable by a hypodermic injection needle). In some aspects, at least one active agent is a lipid nanoparticle or pharmaceutical composition as described herein.

In some aspects, the kits further comprise additional components such as buffers and interpretive information. In some aspects, the kit comprises a container and a label or package insert(s) on or associated with the container. In some aspects, the disclosure provides articles of manufacture comprising the contents of the kits described herein.

General Techniques

The practice of the present disclosure will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Giffiths, and D. G. Newell, eds., 1993-8) J. Wiley and Sons; Method of Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987): PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanette and J. D. Capra, eds., Harwood Academic Publishers, 1995). Without further elaboration, it is believed that one skilled in the art can, based on the above description, utilize the present disclosure to its fullest extent. All publications cited herein are incorporated by reference in their entirety.

### EXAMPLES

### Example 1: Construction of Modified RNAs

To construct the modified RNAs disclosed herein, the following materials and methods were used:

Template Preparation

For replicon RNA, a VEE replicon vector containing the payload was prepared. Methods of preparing such vectors are known in the art. The vector plasmid was further linearized using I-SceI as follows. Briefly, 1 μg of replicon plasmid vector was treated with I-SceI in CutSmart buffer for 1 hour at 37° C. Then, the enzyme was heat inactivated at 65° C. for 20 minutes. The concentration and volume of the different components are provided in Table 3 (below).

TABLE 3
Vector Plasmid Linearization

| Component | Volume (uL) | Concentration | |
|---|---|---|---|
| DNA template | | 1 | μg |
| Cutsmart buffer | 5 | 1× | |
| I-SceI | 1 | 5 | units |

| | |
|---|---|
| Water | 44 |

For modified RNA (modRNA) template, the DNA vector was generated using a replicon plasmid with forward primer containing T7 promoter (TAA TAC GAC TCA CTA TA ATG GAC TAC GAC ATA GT; SEQ ID NO: XX) and SGP and a reverse primer in the 3′-UTR (GAA ATA TTA AAA ACA AAA TCC GAT TCG GAA AAG AA; SEQ ID NO: XX). The Tₘ for the forward and reverse primers were 68° C. and 64° C., respectively. Tables 4 and 5 (below) provide additional information relating to the PCR reaction.

TABLE 4
PCR Setup

| | 25 μl | | FINAL | |
|---|---|---|---|---|
| COMPONENT | REACTION | | CONCENTRATION | |
| 10 μM Forward Primer | 1.25 | μl | 0.5 | μM |
| 10 μM Reverse Primer | 1.25 | μl | 0.5 | μM |
| Template DNA | variable | | 10 | ng |
| 2× Q5 Hot Start Master mix | 12.5 | μl | | |
| Nuclease-Free Water | to 25 | μl | | |

TABLE 5
PCR Cycling Conditions

| STEP | TEMP | | TIME | |
|---|---|---|---|---|
| Initial Denaturation | 98° | C. | 30 | seconds |
| 30 Cycles | 98° | C. | 10 | seconds |
| | 65° | C. | 15 | seconds |
| | 72° | C. | 4 | minutes |
| Final Extension | 72° | C. | 4 | minutes |
| Hold | 4-10° | C. | | |

Plasmid DNA (template) in the PCR reaction was digested by DpnI. More specifically, 1 μL DpnI per μg of initial plasmid was added to the PCR sample and incubated for 1 hour at 37° C.

The PCR (modRNA template) and I-SceI treated replicon DNA (repRNA template) was checked on pre-cast gels to confirm the purity (PCR) and integrity (replicon template) of the replicated construct. Specifically, 20 ng of the DNA was loaded onto a 1.2% DNA gel and ran at 275V for 7-10 minutes. Once confirmed, the DNA was eluted in 20 μL of water.

In Vitro Transcription

To transcribe the above DNA to RNA, a HiScribe High yield T7 kit (New England Biolabs) was used with the modifications described herein. For modified RNA (modRNA) synthesis, the UTP component of the kit was replaced with N1-methylpseudouridine-5′-triphosphate. To begin the in vitro transcription process, the kit components were thawed on ice, mixed, and pulse-spinned in a microfuge. The sample was placed on ice until further use.

Co-transcriptional capping method: For production using cap analog replicon plasmids and modRNA templates, the components shown in Table 6 (below) were mixed, pulse-spun in a microfuge, and then incubated at 37° C. for three hours in the Thermomixer at 400 rpm. A 1 μL aliquot was taken for quality control purposes.

TABLE 6
Co-Transcriptional Capping Components

| Component | Volume | | Final Conc. | |
|---|---|---|---|---|
| Nuclease-free water | X | μl | | |
| 10× Reaction Buffer (NEB) | 2 | μl | 1× | |
| ATP (100 mM) | 2 | μl | 10 | mM final |
| GTP (100 mM) | 2 | μl | 10 | mM final |
| UTP or N1-methylpseudoUTP (100 mM) | 2 | μl | 10 | mM final |
| CTP (100 mM) | 2 | μl | 10 | mM final |
| CleanCap AU | 1 | μl | | |
| Template DNA | X | μl | 1 | μg |
| T7 RNA Polymerase Mix | 2 | μl | | |
| SUPERase Inh. | 1 | μl | | |
| Total reaction volume | 20 | μl | | |

Post-transcriptional enzymatic capping method: For production using enzymatic replicon plasmids, the reaction was assembled at room temperature using the components provided in Table 7 (below).

TABLE 7
Post-Transcriptional Enzymatic Capping Components

| Component | Volume | | Final Conc. | |
|---|---|---|---|---|
| Nuclease-free water | X | μl | | |
| 10× Reaction Buffer | 2 | μl | 1× | |
| ATP (100 mM) | 2 | μl | 10 | mM final |
| GTP (100 mM) | 2 | μl | 10 | mM final |
| UTP or N1-methylpseudoUTP (100 mM) | 2 | μl | 10 | mM final |
| CTP (100 mM) | 2 | μl | 10 | mM final |
| Template DNA | X | μl | 1 | μg |
| T7 RNA Polymerase Mix | 2 | μl | | |
| SUPERase Inh. | 1 | μl | | |
| Total reaction volume | 20 | μl | | |

DNAse treatment: To digest the template DNA, Turbo DNase enzyme was used. There was no need to add the 10× buffer since the enzyme was active in the IVT reactions. The reaction was diluted to 50 μL with nuclease free water. Then, 5 μL of the enzyme (2 U/μL) was added to 20 μL IVT reaction. Then, the mixture was incubated for 30 minutes at 37° C. Afterwards, the RNA was purified using Monarch RNA cleanup kit. A 1 μL aliquot was taken for quality control purposes

Capping and 2′-O-methylation: To prepare a methylated guanine-cap with 2′-O-methylation (Cap 1) structure on the 5′-ends of the IVT mRNAs made using the above-described post-transcriptional capping method, the following method was used. First, the uncapped RNA and nuclease-free water were mixed to a final volume of 13 μL. Then, the mixture was heated at 65° C. for 5 minutes. The mixture was then placed on ice for 5 additional minutes. Then, the components provided in Table 8 (below) were added to the mixture and incubated for 60 minutes at 37° C. Next, the RNA was purified using the small Monarch RNA cleanup kit.

TABLE 8
Capping and 2′-O-Methylation Components

| Component | Volume | |
|---|---|---|
| Denatured uncapped RNA (from above) | 13.0 | μl |
| 10× Capping Buffer | 2.0 | μl |
| GTP (10 mM) | 1.0 | μl |
| SAM (4 mM, dilute 32 mM stock to 4 mM) | 1.0 | μl |
| Vaccinia Capping Enzyme (10 U/μl) | 1.0 | μl |
| mRNA Cap 2′-O-Methyltransferase (50 U/μl) | 1.0 | μl |
| SUPERase Inh | 1.0 | μl |
| Total | 20 | uL |

Poly(A) tail synthesis: To add the poly(A) tails to the modified RNAs, the components provided in Table 9 (below) were added to a reaction tube. Then, the reaction was incubated for 30 minutes at 37° C. Then, the reaction was stopped by directly purifying the RNA with the small Monarch cleanup kit. As a quality control, 200 ng of the RNA was run on a 1.2% RNA gel to confirm the size of the RNA. To do so, RNA was denatured with 50% formaldehyde sample buffer for 5 minutes at 65° C., and then, immediately placed on ice for at least one minute. Then, the denatured RNA was loaded onto a gel and visualized using a Transilluminator.

TABLE 9
Poly(A) tail synthesis components

| Component | Volume |
|---|---|
| RNA | 1-10 μg in 15 μl nuclease free water |
| 10× E. coli Poly(A) Polymerase | 2 μl (1×) |
| Reaction Buffer | |
| ATP (10 mM) | 2 μl |
| E. coli Poly(A) Polymerase | 1 μl |
| SUPERase In | 1 μl |
| Total | 20 μl |

### Example 2: In Vitro Analysis of Expression Kinetics

To assess the expression efficiency of the RNA constructs disclosed herein, both a self-replicating mRNA (repRNA) and a modified mRNA (modRNA) encoding IL-12 protein was constructed using methods described herein (see, e.g., Example 1). Then, the mRNA constructs were transfected into two different cells lines (i.e., B16.F10 and 4T1) using messengerMAX, and the expression level of the encoded protein was assessed at 24, 48, and 72 hours post-transfection.

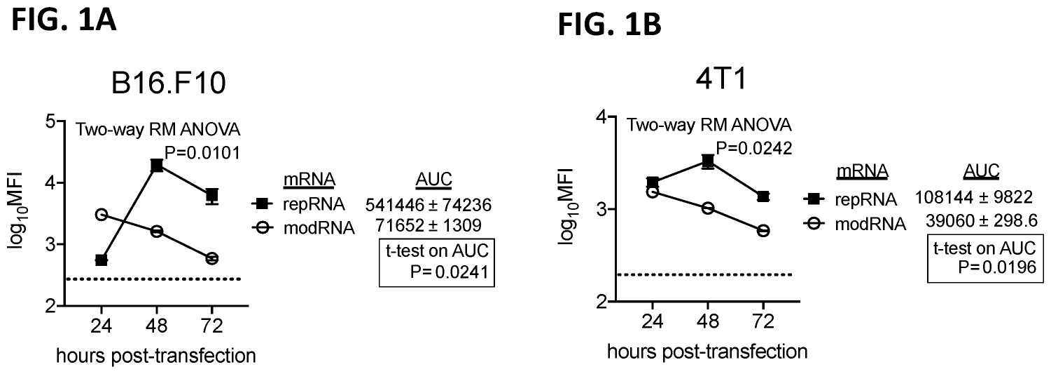

As shown in FIG. 1A, IL-12 expression was observed as early as 24 hours in B16.F10 cells transfected with either the repRNA or the modRNA. By 48 hours post-transfection, a significant difference in IL-12 expression was observed in B16.F10 cells transfected with the repRNA compared to cells transfected with the modRNA. The increased IL-12 expression persisted at least until 72 hours post-transfection. Similar results were observed in the 4T1 cell line (see FIG. 1B). These results suggest that the self-replicating mRNAs disclosed herein are capable of expressing the encoded protein are capable of expressing the encoded protein at higher levels and for longer duration than the modified mRNAs.

### Example 3: In Vivo Analysis of Anti-Tumor Efficacy

To assess whether the RNA constructs disclosed herein can exert activity in vivo, a mouse model of melanoma was used. Briefly, melanoma was induced by inoculating the animals with B6-F10 cells (via subcutaneous administration). Once the tumor reached an optimal size (˜350 mm³), a single high dose of one of the following was injected into the tumors of the animals: administration of one of the following: (i) control mRNA; (ii) modified mRNA encoding IL-12 (modRNA-IL12); and (iii) self-replicating mRNA encoding IL-12 (repRNA-IL12). Then, both tumor volume and survival of the animals were assessed at various time points post treatment.

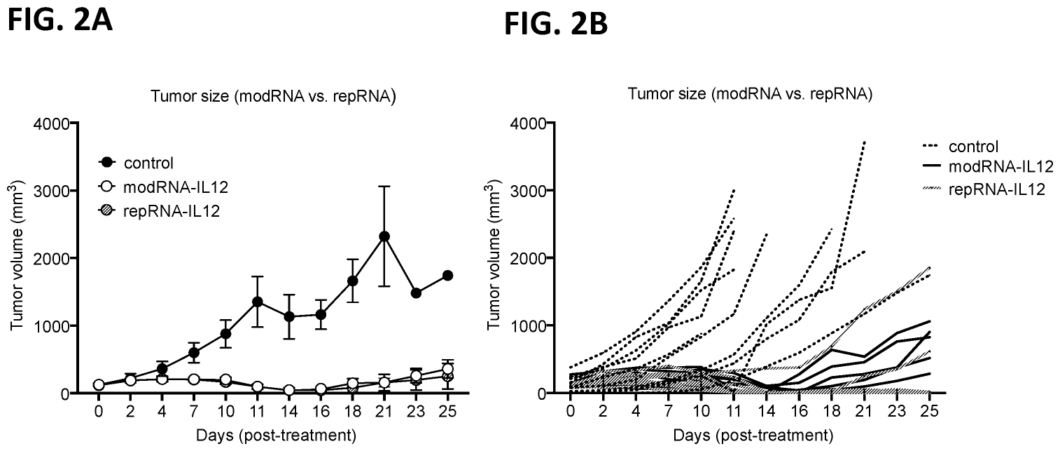

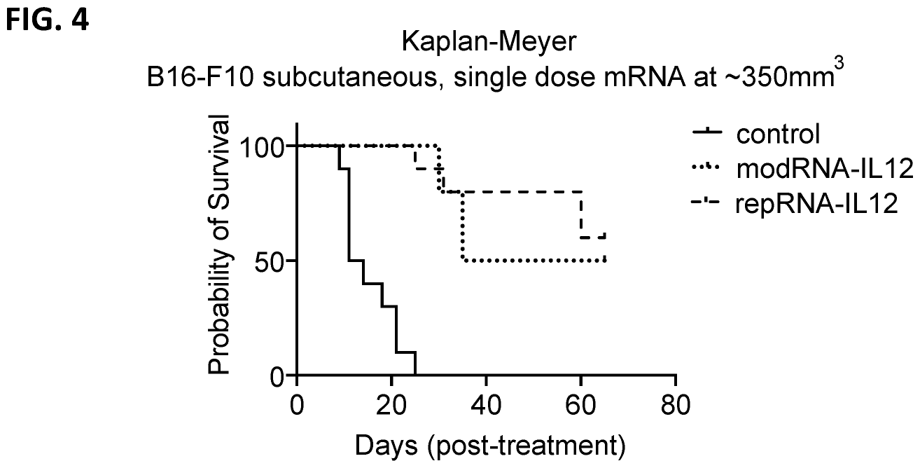

As expected, animals that were treated with the control mRNA failed to control the tumor (see FIGS. 2A and 2B). All the control animals succumbed to the tumor by about day 25 post-treatment (see FIG. 4). In contrast, there was a significant tumor reduction in animals treated with either the modRNA-IL12 or the repRNA-IL12. The overall reduction in tumor volume was similar between the two groups. Animals treated with the repRNA-IL12 construct had slightly greater survival compared to animals that received the modRNA-IL-12 (see FIG. 4). These results suggest that, at least in vivo, the modified mRNA constructs disclosed herein (e.g., encoding IL-12) are nearly as effective as the self-replicating mRNAs (e.g., encoding IL-12) in treating tumor when administered at high doses.

### Example 4: Comparison of Payload Expression after In Vivo Delivery of Self-Replicating mRNA and Modified mRNA

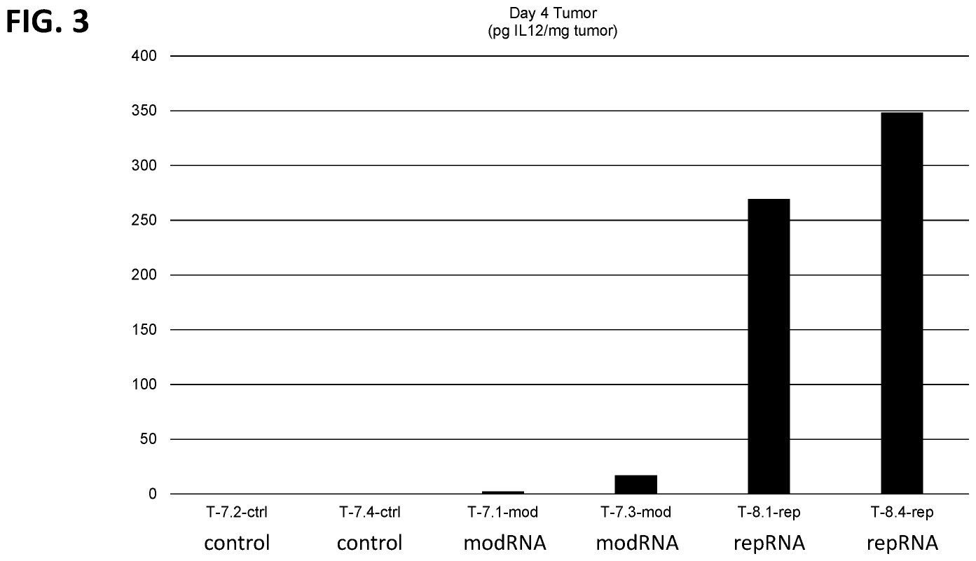

To further characterize the in vivo activity of the RNA constructs disclosed herein, the expression of the encoded IL-12 protein was assessed in the tumor animals from Example 3. As shown in FIG. 3, at day 4 post-treatment, compared to the control, animals treated with either the repRNA-IL12 or the modRNA-IL12 expressed higher levels of IL-12 at the delivery site (i.e., tumor). However, IL-12 expression was significantly higher in animals treated with the modRNA-IL12. This result appears to be consistent with the in vitro data provided in Example 2, suggesting that the self-replicating mRNA constructs disclosed herein are much more efficient at expressing the encoding protein compared to the modified mRNA constructs.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. A lipid nanoparticle comprising (i) one or more types of lipid; and (ii) a modified mRNA comprising a sequence that encodes an interleukin (IL)-12 molecule; wherein the lipid nanoparticle is capable of triggering immunogenic cell death.

2. The lipid nanoparticle of claim 1, wherein the one or more types of lipid comprises a cationic lipid.

3. The lipid nanoparticle of claim 1 or 2, wherein the cationic lipid is a compound of Formula I: 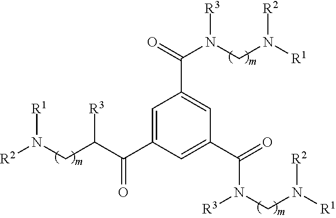
and salts thereof; wherein each R¹ is independently unsubstituted alkyl; each R² is independently unsubstituted alkyl; each R³ is independently hydrogen or substituted or unsubstituted alkyl; and each m is independently 3, 4, 5, 6, 7, or 8.

4. The lipid nanoparticle of claim 3, wherein at least one R¹ is C₁₁H₂₃.

5. The lipid nanoparticle of claim 3 or 4, wherein at least one R³ is hydrogen.

6. The lipid nanoparticle of any one of claims 3 to 5, wherein at least one m is 3.

7. The lipid nanoparticle of any one of claims 3 to 6, wherein each R¹ is independently unsubstituted alkyl; each R2 is independently unsubstituted alkyl, each R³ is hydrogen; and each m is 3.

8. The lipid nanoparticle of any one of claims 2 to 7, wherein the cationic lipid is N1,N3,N5-tris(3-(didodecylamino)propyl)benzene-1,3,5-tricarboxamide (TT3), having the structure: 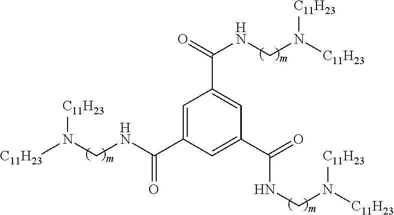
and salts thereof, wherein m is 3.

9. The lipid nanoparticle of any one of claims 2 to 8, wherein the lipid nanoparticle comprises TT3, 1,2-Dioleoyl-sn-glycero-3-phosphoethanolamine (DOPE), cholesterol, and C14-PEG2000.

10. The lipid nanoparticle of any one of claims 1-9, wherein the modified RNA comprises a modified 5′-cap.

11. The lipid nanoparticle of claim 10, wherein the modified 5′-cap is selected from the group consisting of m₂^{7,2′-O}GppspGRNA, m⁷GpppG, m⁷Gppppm⁷G, m₂^{(7,3′-O)}GpppG, m₂^{(7,2′-O)}GppspG(D1), m₂^{(7,2′-O)}GppspG(D2), m₂^{7,3′-O}Gppp(m₁^{2′-O}) ApG, (m⁷G-3′ mppp-G; which may equivalently be designated 3′ O-Me-m7G(5′)ppp(5′)G), N7,2′-O-dimethyl-guanosine-5′-triphosphate-5′-guanosine, m7Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, and combinations thereof.

12. The lipid nanoparticle of any one of claims 1-9, wherein the modified RNA is circular RNA.

13. The lipid nanoparticle of any one of claims 1-12, wherein the modified RNA further comprises a half-life extending moiety.

14. The lipid nanoparticle of any one of claims 1-13, wherein the half-life extending moiety comprises an Fc, an albumin or a fragment thereof, an albumin binding moiety, a PAS sequence, a HAP sequence, transferrin or a fragment thereof, an XTEN, or any combinations thereof.

15. The lipid nanoparticle of any one of claims 1-14, wherein the IL-12 molecule is selected from the group consisting of IL-12, an IL-12 subunit, or a mutant IL-12 molecule that retains the immunomodulatory function.

16. The lipid nanoparticle of claim 15, wherein the IL-12 comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 1.

17. The lipid nanoparticle of claim 16, wherein the IL-12 molecule comprises IL-12a and/or IL-120 subunits.

18. The lipid nanoparticle of claim 17, wherein the IL-12a subunit comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 2.

19. The lipid nanoparticle of claim 17, wherein the IL-120 subunit comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 3.

20. The lipid nanoparticle of claim 17, wherein the IL-12a subunit and the IL-120 subunit are linked by a linker.

21. The lipid nanoparticle of claim 20, wherein the linker comprises an amino acid linker of at least about 2, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 amino acids.

22. The lipid nanoparticle of claim 21, wherein the linker comprises a (GS) linker.

23. The lipid nanoparticle of claim 22, wherein the GS linker has a formula of (Gly4Ser)n or S(Gly4Ser)n, wherein n is a positive integer selected from the group consisting of 1, 2, 3, 4, 5, 6, 7, 8, 9, 10, 11, 12, 13, 14, 15, 16, 17, 18, 19, 20, 30, 40, 50, 60, 70, 80, or 100.

24. The lipid nanoparticle of claim 23, wherein the (Gly4Ser)n linker is (Gly4Ser)3 or (Gly4Ser)4.

25. The lipid nanoparticle of any one of claims 1-24, wherein the IL12 molecule comprises an amino acid sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 4 or SEQ ID NO: 5

26. The lipid nanoparticle of any one of claims 1-25, wherein the modified mRNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 6

27. The lipid nanoparticle of any one of claims 1-25, wherein the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 7.

28. The lipid nanoparticle of any one of claims 1-27, wherein the modified RNA further comprises a regulatory element.

29. The lipid nanoparticle of claim 28, wherein the regulatory element is selected from the group consisting of at least one translation enhancer element (TEE), a translation initiation sequence, at least one microRNA binding site or seed thereof, a 3′ tailing region of linked nucleosides, an AU rich element (ARE), a post transcription control modulator, and combinations thereof.

30. The lipid nanoparticle of claim 29, wherein the 3′ tailing region of linked nucleosides comprises a poly-A tail, a polyA-G quartet, or a stem loop sequence.

31. The lipid nanoparticle of any one of claims 1-30, wherein the modified RNA comprises at least one modified nucleoside.

32. The lipid nanoparticle of claim 31, wherein the at least one modified nucleoside is selected from the group consisting of 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, and combinations thereof.

33. The lipid nanoparticle of any one of claims 1-32, wherein the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 8

34. The lipid nanoparticle of any one of claims 1-32, wherein the modified RNA comprises a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 9.

35. The lipid nanoparticle of any one of claims 1-34, wherein the lipid nanoparticle has a diameter of about 30-500 nm.

36. The lipid nanoparticle of any of claims 1-35, wherein the lipid nanoparticle has a diameter of about 50-400 nm.

37. The lipid nanoparticle of any one of claims 1-36, wherein the lipid nanoparticle has a diameter of about 70-300 nm.

38. The lipid nanoparticle of any one of claims 1-37, wherein the lipid nanoparticle has a diameter of about 100-200 nm.

39. The lipid nanoparticle of any one of claims 1-38, wherein the lipid nanoparticle has a diameter of about 100-175 nm

40. The lipid nanoparticle of any one of claims 1-39, wherein the lipid nanoparticle has a diameter of about 100-120 nm.

41. The lipid nanoparticle of any one of claims 1-40, wherein the lipid nanoparticle and the modified RNA have a mass ratio of about 1:2 to about 2:1.

42. The lipid nanoparticle of claim 41, wherein the lipid nanoparticle and the modified RNA have a mass ration of 1:2, 1:1.5, 1:1.2, 1:1.1, 1:1, 1.1:1, 1.2:1, 1.5:1, or 2:1.

43. The lipid nanoparticle of claim 42, wherein the lipid and the modified RNA have a mass ratio of about 1:1.

44. A pharmaceutical composition, comprising the lipid nanoparticle of any one of claims 1-43 and a pharmaceutically acceptable carrier.

45. The pharmaceutical composition of claim 44, wherein the pharmaceutical composition is formulated for intratumoral, intrathecal, intramuscular, intravenous, subcutaneous, inhalation, intradermal, intralymphatic, intraocular, intraperitoneal, intrapleural, intraspinal, intravascular, nasal, percutaneous, sublingual, submucosal, transdermal, or transmucosal administration.

46. A method for treating cancer in a subject in need thereof, comprising administering to the subject an effective amount of the lipid nanoparticle of any one of claims 1-43 or the pharmaceutical composition of claim 44 or 45.

47. The method of claim 46, wherein the subject is a human patient having or suspected of having a cancer.

48. The method of claim 47 wherein the human patient has a cancer selected from the group consisting of melanoma, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine cancer, salivary gland carcinoma, kidney cancer, prostate cancer, vulval cancer, thyroid cancer, hepatic carcinoma, gastric cancer, and head and neck cancer.

49. The method of any one of claims 46-48, wherein the lipid nanoparticle or the pharmaceutical composition is administered to the subject in a single dose.

50. The method of any one of claims 46-49, wherein the pharmaceutical composition is administered to the subject by intratumoral injection, intramuscular injection, subcutaneous injection, or intravenous injection.
